SEQUENCE LISTING (15 sequences)

SEQ ID NO: 1 - DNA, 41 bp, Artificial Sequence
  Description of Artificial Sequence Synthetic primer
taatacgactcactatagggcggcctcttcttatttatggc

SEQ ID NO: 2 - DNA, 23 bp, Artificial Sequence
  Description of Artificial Sequence Synthetic primer
gggcttgcctgatttgcccatac

SEQ ID NO: 3 - DNA, 40 bp, Artificial Sequence
  Description of Artificial Sequence Synthetic primer
taatacgactcactatagggcttgcctgatttgcccatac

SEQ ID NO: 4 - DNA, 24 bp, Artificial Sequence
  Description of Artificial Sequence Synthetic primer
gggcggcctcttcttatttatggc

SEQ ID NO: 5 - DNA, 22 bp, Artificial Sequence
  Description of Artificial Sequence Synthetic primer
gggcgccatgataatgttggac

SEQ ID NO: 6 - DNA, 39 bp, Artificial Sequence
  Description of Artificial Sequence Synthetic primer
taatacgactcactatagggcgccatgataatgttggac

SEQ ID NO: 7 - DNA, 21 bp, Artificial Sequence
  Description of Combined DNA/RNA Molecule Synthetic oligonucleotide
cuuacgcugaguacuucgatt

SEQ ID NO: 8 - DNA, 21 bp, Artificial Sequence
  Description of Combined DNA/RNA Molecule Synthetic oligonucleotide
ucgaaguacucagcguaagtt

SEQ ID NO: 9 - DNA, 21 bp, Artificial Sequence
  Description of Combined DNA/RNA Molecule Synthetic oligonucleotide
uucuccgaacgugucacgutt

SEQ ID NO: 10 - DNA, 21 bp, Artificial Sequence
  Description of Combined DNA/RNA Molecule Synthetic oligonucleotide
acgugacacguucggagaatt

SEQ ID NO: 11 - protein, 827 aa, Homo sapiens
MRGCLRLALLCALPWLLLAASPGHPAKSPRQPPAPRRDPFDAARGADFDHVYSGVVNLSTENIYSFNYTSQPDQVTAVRVYVNSSSENLNYPVLVVVRQQKEVLSWQVPLLFQGLYQRSYNYQEVSRTLCPSEATNETGPLQQLIFVDVASMAPLGAQYKLLVTKLKHFQLRTNVAFHFTASPSQPQYFLYKFPKDVDSVIIKVVSEMAYPCSVVSVQNIMCPVYDLDHNVEFNGVYQSMTKKAAITLQKKDFPGEQFFVVFVIKPEDYACGGSFFIQEKENQTWNLQRKKNLEVTIVPSIKESVYVKSSLFSVFIFLSFYLGCLLVGFVHYLRFQRKSIDGSFGSNDGSGNMVASHPIAASTPEGSNYGTIDESSSSPGRQMSSSDGGPPGQSDTDSSVEESDFDTMPDIESDKNIIRTKMFLYLSDLSRKDRRIVSKKYKIYFWNIITIAVFYALPVIQLVITYQTVVNVTGNQDICYYNFLCAHPLGVLSAFNNILSNLGHVLLGFLFLLIVLRRDILHRRALEAKDIFAVEYGIPKHFGLFYAMGIALMMEGVLSACYHVCPNYSNFRFDTSFMYMIAGLCMLKLYQTRHPDINASAYSAYASFAVVIMVTVLGVVFGKNDVWFWVIFSAIHVLASLALSTQIYYMGRFKIDLGIFRRAAMVFYTDCIQQCSRPLYMDRMVLLVVGNLVNWSFALFGLIYRPRDFASYMLGIFICNLLLYLAFYIIMKLRSSEKVLPVPLFCIVATAVMWAAALYFFFQNLSSWEGTPAESREKNRECILLDFFDDHDIWHFLSATALFFSFLVLLTLDDDLDVVRRDQIPVF

SEQ ID NO: 12 - protein, 832 aa, Homo sapiens
MRGCLRLALLCALPWLLLAASPGHPAKSPRQPPAPRRDPFDAARGADFDHVYSGVVNLSTENIYSFNYTSQPDQVTAVRVYVNSSSENLNYPVLVVVRQQKEVLSWQVPLLFQGLYQRSYNYQEVSRTLCPSEATNETGPLQQLIFVDVASMAPLGAQYKLLVTKLKHFQLRTNVAFHFTASPSQPQYFLYKFPKDVDSVIIKVVSEMAYPCSVVSVQNIMCPVYDLDHNVEFNGVYQSMTKKAAITLQKKDFPGEQFFVVFVIKPEDYACGGSFFIQEKENQTWNLQRKKNLEVTIVPSIKESVYVKSSLFSVFIFLSFYLGCLLVGFVHYLRFQRKSIDGSFGSNDGSGNMVASHPIAASTPEGSNYGTIDESSSSPGRQMSSSDGGPPGQSDTDSSVEESDFDTMPDIESDKNIIRTKMFLYLSDLSRKDRRIVSKKYKIYFWNIITIAVFYALPVIQLVITYQTVVNVTGNQDICYYNFLCAHPLGVLSAFNNILSNLGHVLLGFLFLLIVLRRDILHRRALEAKDIFAVEYGIPKHFGLFYAMGIALMMEGVLSACYHVCPNYSNFQFDTSFMYMIAGLCMLKLYQTRHPDINASAYSAYASFAVVIMVTVLGVVFGKNDVWFWVIFSAIHVLASLALSTQIYYMGRFKIDVSDTDLGIFRRAAMVFYTDCIQQCSRPLYMDRMVLLVVGNLVNWSFALFGLIYRPRDFASYMLGIFICNLLLYLAFYIIMKLRSSEKVLPVPLFCIVATAVMWAAALYFFFQNLSSWEGTPAESREKNRECILLDFFDDHDIWHFLSATALFFSFLVLLTLDDDLDVVRRDQIPVF

SEQ ID NO: 13 - DNA, 4669 bp, Homo sapiens
tttttattattgctgttattgaggttgagggagaagagatcggtctaaattctggctgggtaagtggggggattctcggcgatgagaaacgggggacttagaagccggaggaaaatcagcagccccacatctccacttctccagtccgccctactctccacccgtgacctccagtggagaccccaggcggcagcatcagtatttgatcggcccttcgtcagcacgctgccagccctggccggctgggttcgccaggcatcacccgctcggctctgaagcggacgcctggccctgcaccgggctttggaaggaccctctctgcgctcgccccctccccagggtggctccgctttcgagcccgggcgcggcgcccaccatgcgcggctgcctgcggctcgcgctgctctgcgcgctgccctggctcctgctggcggcgtcgcccgggcacccggcgaaatcccccaggcagcccccggcaccgcgccgcgaccccttcgacgctgccaggggcgccgatttcgatcatgtctacagcggggtggtgaacctcagcaccgagaacatctactctttcaactacaccagccagcccgaccaggtgacagccgtgagggtgtatgtgaacagttcctctgagaatctcaactacccggtccttgttgtggttcgccagcagaaagaggtgctgtcctggcaggttcctctgctcttccaaggactataccagaggagctacaattatcaagaagtgagccgcaccttatgtccctcagaagcaaccaatgagacgggacccttgcagcaactgatatttgtagatgtcgcatccatggcacccctgggtgctcagtacaaactgctagttaccaagctgaagcacttccagctccggacaaatgttgcctttcactttactgccagcccctctcaacctcagtattttctatacaagtttcccaaagacgtggactcagttatcattaaagtggtgtctgaaatggcttatccatgttctgttgtctcagtccagaatatcatgtgcccggtgtatgatctcgaccacaatgtggaatttaatggtgtctatcagtccatgaccaagaaagctgccatcacgctacagaagaaggattttccaggcgagcagttcttcgtggtatttgtgataaagcctgaagattatgcctgtggaggatctttcttcatccaggaaaaggaaaaccagacctggaatctacagcgaaaaaagaaccttgaagtgaccattgtcccttccattaaagaatctgtttatgtgaaatccagtcttttcagtgtcttcatcttcctgtccttctacttgggatgccttcttgttgggtttgttcattatctgaggtttcagagaaaatccattgatggaagctttgggtccaatgatggctctggaaatatggtggcatctcatcccattgctgccagcacacccgaagggagcaattatgggacaatagatgagtcaagctccagtcctggaaggcagatgtcctcctccgatggtgggccaccgggccagtcagacacagacagctccgtggaggagagcgacttcgacaccatgccagacattgagagtgataaaaacatcatccggaccaagatgttcctttacctgtcagatttgtccaggaaggaccggagaattgtcagcaaaaaatataaaatttatttttggaacatcatcaccattgctgtgttttacgcgctgcccgtgatccagctggtcattacctatcagacagtggtaaatgtcactggcaaccaggacatctgttactacaacttcctctgtgctcaccccttgggcgtcctgagtgccttcaacaacattctcagcaatctgggccacgtgcttctgggcttcctcttcctgctgatagtcttgcgccgcgacatcctccatcggagagccctggaagccaaggacatctttgctgtggagtacgggattcccaaacactttggtctcttctacgctatgggcattgcattgatgatggaaggggtgctcagtgcttgctaccatgtctgccctaattattccaacttccgattcgacacctccttcatgtacatgatcgctggcctgtgcatgctgaagctctatcagacccgccacccagacatcaatgccagcgcctactctgcctatgcctcctttgctgtggtcatcatggtcaccgtccttggagtggtgtttggaaaaaatgacgtatggttctgggtcatcttctctgcaatccacgttctggcctcgctagccctcagcacccagatatattatatgggtcgtttcaagatagatttgggaattttccggcgggctgccatggtgttctacacagactgtatccagcagtgtagccgacctctatatatggatagaatggtgttgctggttgtggggaatctggttaactggtccttcgccctctttggattgatataccgccccagggactttgcttcctacatgctgggcatcttcatctgtaaccttttgctgtacctggccttttacatcatcatgaagctccgcagctctgaaaaggtcctcccagtcccgctcttctgcatcgtggccaccgctgtgatgtgggctgccgccctatattttttcttccagaatctcagcagctgggagggaactccggccgaatcccgggagaagaaccgcgagtgcattctgctggatttcttcgatgaccatgacatctggcacttcctctctgctactgctctgtttttctcattcttggttttgttaactttggatgatgaccttgatgtggttcggagagaccagatccctgtcttctgaacctccaacattaagagaggggagggagcgatcaatcttggtgctgtttcacaaaaattacagtgaccacagcaaagtaaccactgccagatgctccactcaccctctgtagagccaactctgcattcacacaggaaggagaggggctgcgggagatttaaacctgcaagaaaggaggcagaaggggagccatgttttgaggacagacgcaaacctgaggagctgagaaacacttgctccttccatctgcagctttgggagtgcaacagggataggcactgcatccaagtcaactcaccatcttggggtccctcccaccctcacggagacttgccagcaatggcagaatgctgctgcacacttccctccagttgtcaccctgcccagaaaggccagcagcttggacttcctgcccagaaactgtgttggcccccttcacacctctgcaacacctgctgctccagcaagaggatgtgattctttagaatatggcggggaggtgaccccaggccctgccctactgggatagatgttttaatggcaccagctagtcacctcccagaagaaactctgtacatttcccccaggtttctgatgccatcagaagggctcaggagtggggtttgtcacacattcctcttaacaagtaactgtcactgggaccgagtcctgggtgcttacatattccttcgtgtcttcatctcactgacctgtgtggacctcatcactctgactctgccttcttggaaaggccctgtcactccacagatgtctggccagcttcaaggcagaaggaaaaacaggaaaagctcttttaacagcagcaggaacaagagaaatgactaaccatactaaaagactggtaacagcagcagcagccagacaggcctcaccttaaggacttgggctgccagagcaaattcagcagagcttatttggcctcccattcacacagctcagttctgtgcccacatcacctttggggaagaaatcagcattctaatcagggacactacttcaggagtcctccacagcgagtccgtcatctgtcactttatgtagatcagggttctagacttcttccctgaggttctcagaagcagctctcaggatgaacgtattgtcctcttcccctcttcttgcaaagtgcacagctaatctaatgttgtctctcggttgcacctgacattctctccccagtaaggtgttggcaagctcagcatctgggttccactctcacactgtctggcagctctgtgtctgagaagttctacattgaccaggcccccttgttgcctggagtatgacgtaatcagaaaatagacgtataaatgtgcacatgcgtatgtatttgcttgtgaaattaaagtcacctcttgcctctgctttcctgatcattcgttagagaaatggatcaggcatttttttaaattattattctttctctaaactatttgcattgtgttcaaaaacccattttagaagtttgaacagcaagcttttcctgattttaaaaacacaaagttgctttcaatgaaatattttgtgatttttttaaagtccccaaatgtgtacttagccttctgttattccttattctttaagcagtgttggcttccattgaccatatgaaggccaccaattaaatggttgtgttaatccaacatgtaaaaaactttttggcagggcacagtggctcacgcctgtaatcccaaagtgctggaatttacaggtaggaccaccacacttggtccacttacttataataaacattgatttggtcgttaaaaaaaaaaaaaaaaaaaaaaa

SEQ ID NO: 14 - DNA, 2672 bp, Caenorhabditis elegans
ggtttaattacccaagtttgaggttcacaatgattcgtgtttatttgataattttaatgcatttggtgattggtttaacccagaacaattcaactacaccttcgccaattatcacctcaagtaacagctctgtacttgtattcgagatttcttcaaaaatgaaaatgatcgaaaaaaagctggaagccaacacagtccatgtccttcgcctggaattagatcaaagtttcatattagatttaaccaaagtcgccgcggaaatcgttgattcttcgaaatacagtaaagaagacggtgttatactcgaagtaacagtttcaaatggccgtgatagttttttattgaaacttccgacggtttatccgaacttgaagctctatactgacggaaaactgctcaatccgctcgttgagcaagatttcggggcgcacagaaagaggcacaggataggcgaccctcatttccatcaaaacctgatcgtaaccgtgcagtctcgattgaatgctgatatagattataggcttcatgtgactcatttggatcgggcccaatatgattttctgaagttcaagacgggacagaccacgaaaacgttgtcgaatcagaagctgacgtttgtcaagccgattggattttttttgaattgcagcgaacaaaatatttcccaattccacgtcacattgtacagtgaagatgatatttgtgcaaatctgataactgtgccggcgaatgaatccatctatgatcgatcagtgatttccgataaaactcacaatcgacgtgtcctatcattcaccaaaagagccgacatttttttcactgaaactgaaatctcgatgttcaaatcattccgaatcttcgtcttcatagctcccgatgattctggatgttctaccaacacatcacgcaaaagtttcaacgagaaaaagaaaatatcttttgaattcaaaaaactggaaaatcaatcatacgccgtcccgacggctttgatgatgatatttctgacgacaccgtgtcttttgttccttccaattgtgattaatattatcaagaatagcagaaaattggcaccatcacaatcaaatcttatctcattttctccagttccgtctgagcaacgggacatggatttgagccatgatgagcagcagaatacgagctcagaactcgaaaataatggagaaattccagcagcagaaaatcaaattgttgaagagatcacggctgaaaatcaagaaacgagcgtagaagagggaaaccgggaaattcaagttaaaattccgttgaaacaggattcattatcactccatggccaaatgcttcaatatcccgttgcaataattctcccagttctcatgcacacagctatcgaattccataaatggacgacatctacaatggcaaatcgcgacgaaatgtgcttccacaatcatgcgtgtgctcggccattgggagaacttcgagcttggaataatatcatcaccaatataggatatactctttatggagccatcttcattgttttgtcgatatgtagaagaggccgtcatgagtattctcatgtttttggtacatatgaatgcacacttttagatgtgactattggtgttttcatggttttgcaatcaattgctagtgccacttatcatatttgccccagtgatgtggcttttcagtttgatacgccgtgcatccaagttatctgtggacttctcatggtccgtcagtggtttgttcgtcacgaatctccatcaccagcctatacaaatatcctactagttggagttgtctccttgaactttctaatatctgcattctccaaaacatcatatgtccgattcatcatcgctgtaattcatgtcattgtcgttggatcgatctgtttggcaaaggaaagatccttgggatcggaaaaattaaaaactcgatttttcatcatggccttctcgatgggaaatttcgcagcaatcgtgatgtatctgacgctttcggcgtttcatttgaatcaaatagccacgtattgctttattataaattgtatcatgtatctgatgtactatggatgcatgaaagttttacatagcgagagaataacgtcgaaggctaaactttgtggagctctgtcactgctcgcgtgggctgttgccggatttttcttctttcaagatgatacagattggacgagatctgcggcggcgagccgagcactcaacaagccatgcctgctactcggcttcttcggttcccacgatttatggcacatcttcggagcattggccggtcttttcacattcattttcgtctcctttgttgatgatgatctcattaatacacgcaaaacttcgattaacattttctagagtaattttgttttccctatccggtttttcatatttcaactttttctcctgccttaatacgtagcccgatctctcatttcttcatgttttaagaactttctgaatctatgtaattagttggtctactttgaaacatttttactgtattcatctccacggctccctacctccctgtgaatataattccaaatttagctcctctctacgcagtgatctggtgtttataacatgtttatttatttttcccaacatttcccaatttcaggtgttcacttgtttgtttgctccccctctatgcataaattttcagcc

SEQ ID NO: 15 - protein, 718 aa, Caenorhabditis elegans
MIRVYLIILMHLVIGLTQNNSTTPSPIITSSNSSVLVFEISSKMKMIEKKLEANTVHVLRLELDQSFILDLTKVAAEIVDSSKYSKEDGVILEVTVSNGRDSFLLKLPTVYPNLKLYTDGKLLNPLVEQDFGAHRKRHRIGDPHFHQNLIVTVQSRLNADIDYRLHVTHLDRAQYDFLKFKTGQTTKTLSNQKLTFVKPIGFFLNCSEQNISQFHVTLYSEDDICANLITVPANESIYDRSVISDKTHNRRVLSFTKRADIFFTETEISMFKSFRIFVFIAPDDSGCSTNTSRKSFNEKKKISFEFKKLENQSYAVPTALMMIFLTTPCLLFLPIVINIIKNSRKLAPSQSNLISFSPVPSEQRDMDLSHDEQQNTSSELENNGEIPAAENQIVEEITAENQETSVEEGNREIQVKIPLKQDSLSLHGQMLQYPVAIILPVLMHTAIEFHKWTTSTMANRDEMCFHNHACARPLGELRAWNNIITNIGYTLYGAIFIVLSICRRGRHEYSHVFGTYECTLLDVTIGVFMVLQSIASATYHICPSDVAFQFDTPCIQVICGLLMVRQLGSEKLKTRFFIMAFSMGNFAAIVMYLTLSAFHLNQIATYCFIINCIMYLMYYGCMKVLHSERITSKAKLCGALSLLAWAVAGFFFFQDDTDWTRSAAASRALNKPCLLLGFFGSHDLWHIFGALAGLFTFIFVSFVDDDLINTRKTSINIF

(19) United States
(12) Patent Application Publication
(10) Pub. No.: US 2008/0112938 A1
(43) Pub. Date: May 15, 2008

(54) RECOMBINANT EUKARYOTIC CELLS STABLY EXPRESSING (SID-1) PROTEINS FOR HIGH THROUGHPUT GENE SCREENING

(75) Inventors: Ronald Li, Davis, CA (US); Jennifer C. Moore, Easton, PA (US)

Correspondence Address: FOLEY & LARDNER LLP, 975 PAGE MILL ROAD, PALO ALTO, CA, 94304, US

(73) Assignee: The Regents of the University of California

(21) Appl. No.: 11/864,435

(22) Filed: Sep. 28, 2007

Related U.S. Application Data
(60) Provisional application No. 60/848,248, filed on Sep. 28, 2006.

(51) Int. Cl. A61K 48/00; C12Q 1/68; C12N 5/08

(52) U.S. Cl. 424/93.21; 435/455; 435/368; 435/6

(57) ABSTRACT

This invention provides a eukaryotic cell that stably expresses exogenous, ectopic SID-1 to confer enhanced polynucleotide, e.g. siRNA or dsRNA, uptake. Thus, in one aspect, this invention provides a eukaryotic cell which stably expresses exogenous SID-1 polynucleotide and is. The cells stably expressing SID-1 are particularly useful for high throughput screening of gene activity using RNA interference. Methods for producing and using the cells also are provided in this application.

### CROSS-REFERENCE TO RELATED APPLICATIONS

The present application claims the benefit under 35 U.S.C. § 119(e) of U.S. Provisional Ser. No. 60/848,248, filed Sep. 28, 2006, the contents of which is hereby incorporated by reference into the present disclosure.

### STATEMENT OF GOVERNMENT SUPPORT

This invention was supported in whole or in part under the following grant: NIH (RO1 HL72857). Accordingly, the U.S. government may have rights to the inventions disclosed herein.

### BACKGROUND

RNA interference (RNAi), a post-transcriptional gene silencing mechanism originally described in C. elegans and plants, involves sequence-specific degradation of homologous messenger RNA (mRNA) mediated by double-stranded RNA (dsRNA) molecules (see Fire A., et al. (1998) Nature 391:806-811): dsRNAs are cleaved by the conserved Dicer family of RNase III enzymes to produce short interfering RNAs (siRNAs) that are typically 21-23 nucleotides in length. siRNAs then get incorporated into the RNA-induced silencing complex (RISC) where the antisense strand of siRNA directs the recognition and subsequent cleavage of the target mRNA to result in gene-specific silencing. Hannon G. J. (2002) Nature 418:244-251, Meister G., et al. (2004) Nature 431:343-349. Whereas dsRNAs typically enter cells by natural viral infections (which in turn can lead to interferon responses in certain mammalian cell types) or other exogenous means, systemic dsRNA uptake has been uniquely observed in C. elegans (see Svoboda P. (2004) Cytogenet Genome Res 105:422-434) due to expression of systemic RNA interference defective-1 (SID-1), a 776-amino acid transmembrane channel protein. Feinberg E. H., et al. (2003) Science 301:1545-1547, Winston W. M., et al. (2002) Science 295:2456-2459. Interestingly, ectopic expression of the C. elegans protein SID-1 in Drosophila cells enables passive cellular uptake of soaking dsRNA or siRNA. Feinberg E. H., et al. (2003) Science 301:1545-1547.

Although in the experimental setting, such conventional methods as injection, transient transfection (e.g. cationic lipid-based transfection reagent), electroporation and recombinant virus-mediated delivery of short hairpin RNA (shRNAs) are effective for inducing RNAi in mammalian cells, these approaches often require the generation and testing of multiple constructs (especially when multiple genes need to be screened) which can be labor-intensive.

This invention overcomes these limitations and provides related advantages as well.

### SUMMARY OF THE INVENTION

This invention provides a mammalian cell as an example of an eukaryotic cell that stably expresses exogenous, ectopic SID-1 to confer enhanced siRNA or dsRNA uptake in the transfected or transduced cells. Thus, in one aspect, this invention provides a mammalian or a eukaryotic cell transduced with exogenous siRNA or dsRNA polynucleotide and exogenous SID-1 polynucleotide, wherein the cell stably expresses or overexpresses the exogenous SID-1 polynucleotide. In a particular aspect, the eukaryotic cell is a mammalian cell such as a cultured human embryonic kidney cell (HEK 293T). In an alternative embodiment, the mammalian cell is an isolated stem cell, e.g., pluripotent or multipotent stem cells and/or their derivatives. The stem cell can be of human or animal, e.g., mammalian, origin. The transformed cells are particularly useful for high throughput screening of gene activity, particularly during development, such as through the use of RNA interference.

Also provided by this invention is a method for preparing a eukaryotic cell that stably expresses SID-1 polynucleotide and in a further aspect, expresses stably or transiently exogenous siRNA or dsRNA polynucleotide, by passively contacting a stably transformed eukaryotic cell expressing or overexpressing exogenous SID-1 polypeptide with the exogenous siRNA or dsRNA. Although traditional vectors can be used in one aspect to insert exogenous polynucleotide into cells, Applicants have found that passively soaking the cells with the polynucleotide is effective for delivery and expression of the polynucleotides.

Also provided by this invention is a method for inducing gene-specific RNA interference in a cell, by introducing an exogenous SID-1 polynucleotide into a mammalian stem cell and passively contacting the cell with a gene-specific siRNA or dsRNA in the absence of a gene delivery vehicle, vector or carrier such that gene expression for the targeted gene is reduced.

Also provided by this invention is a method for studying stem cell development using RNA interference, by introducing an exogenous SID-1 polynucleotide into mammalian stem cells, passively contacting the cell with a gene-specific siRNA or dsRNA in the absence of an insertion vector or carrier such that gene expression for the targeted gene is reduced, and comparing the development of the stem cell to a stem cell not subjected to RNA interference.

The eukaryotic cells can be used in a method for detecting gene specific interference by an exogenous siRNA or dsRNA and resultant functional consequences by comparing the change in label detected from a cell as described above at least two distinct time points, such as before and after addition of the exogenous siRNA or dsRNA.

The present invention also provides a mammalian stem cell as an example of a eukaryotic cell comprising exogenous siRNA or dsRNA polynucleotide and exogenous SID-1 polynucleotide, wherein the cell stably expresses the exogenous SID-1 polypeptide. In one aspect, the mammalian stem cell is a human stem cell. In a further embodiment, the human stem cell is a somatic stem cell or an embryonic stem cell. In another embodiment, the RNA polynucleotide is transcribed from exogenous DNA polynucleotide. In another embodiment, the SID-1 polynucleotide is DNA polynucleotide or RNA polynucleotide. In another embodiment, the RNA polynucleotide is siRNA. In another embodiment, the polynucleotide, DNA or RNA, further comprises a detectable label. In another embodiment, the RNA polynucleotide is complementary to a gene having biological activity selected from the group consisting of apoptosis, cell adhesion, cell cycle, immunotherapy, cell signaling, DNA repair and DNA synthesis. In another embodiment, the RNA polynucleotide is complementary to a gene having biological activity in immunotherapy. In another embodiment, the gene encodes a cytokine, chemokine, transcriptional regulation factor or a translational regulation factor.

The present invention also provides a method for inducing gene-specific RNA interference in a mammalian stem cell as an example of a eukaryotic or animal stem cell by enhancing endogous expression of SID-1 polynucleotide or alternatively introducing an exogenous SID-1 polynucleotide into a mammalian stem cell and passively contacting the mammalian stem cell with the gene-specific siRNA or dsRNA in the absence of a gene delivery vehicle, vector or carrier such that gene expression for the targeted gene is reduced. For these aspects of the invention, the mammalian stem cell can be a human stem cell. In another embodiment, the human stem cell is a somatic (adult) stem cell or an embryonic stem cell. In another embodiment, the RNA polynucleotide is siRNA. In another embodiment, the RNA polynucleotide is complementary to a gene having biological activity selected from the group consisting of apoptosis, cell adhesion, cell cycle, immunotherapy, cell signaling, DNA repair, DNA synthesis. In another embodiment, the RNA polynucleotide is complementary to a gene having biological activity in immunotherapy. In another embodiment, the gene encodes a cytokine, chemokine, transcriptional regulation factor or a translational regulation factor.

The present invention further provides a method of studying stem cell development using RNA interference by introducing an exogenous SID-1 polynucleotide or enhancing endogenous SID-1 expression in a eukaryotic cell such as a mammalian stem cell. In some aspects, the exogenous polynucleotide is passively contacting the stem cell with the gene-specific siRNA or dsRNA in the absence of a gene delivery vehicle, vector or carrier, such that gene expression for the targeted gene is reduced, and comparing the development of the stem cell to a stem cell not subjected to RNA interference. In another embodiment, more than one gene is silenced by siRNA. In another embodiment, the stem cell is a mammalian stem cell such as a human stem cell. In another embodiment, the human stem cell is a somatic stem cell or an embryonic stem cell. In another embodiment, the method is performed as a high throughput screen. This method and system is useful to screen for agents and compositions that modulate activity of pre-selected proteins and regulatory polynucleotides.

Also provided by this invention are compositions containing SID-1 polynucleotide and optionally polynucleotides encoding siRNA or dsRNA for use in stably expressing SID-1 polynucleotide in eukaryotic cells as described herein.

### BRIEF DESCRIPTION OF THE DRAWINGS

FIG. 1A is a schematic representation of LV-CAG-SID-1-GFP.

FIG. 1B shows a FACS analysis of control, untransduced (left) and HEK cells after LV-CAG-SID-1-GFP transduction (right). Cells that expressed the highest GFP signal, which correspond to 8.23% of the original population, were sorted for subsequent experiments.

FIG. 1C is a representative images (top, green fluorescence; bottom, phase contrast) of LV-CAG-SID-1-GFP-transduced HEK cells after recovery from sorting (from B). Note that GFP epifluorescence was limited to the membrane surface.

FIG. 1D is a Western blot analysis. Specific bands at 116 kDa consistent with the expected size of SID-1-GFP were observed when probed with an anti-GFP antibody.

FIG. 2A is a bar graph summarizing the effects of transient transfection of various siRNA or dsRNA constructs on luc^{Fir} activity. luc^{Fir} expression levels were normalized to that of control cells (luc^{Fir} only). Transfection with luc^{Fir}-siRNA (0.35 and 0.7 μg/ml), 100 bp (1 μg/ml) or 500 bp (0.01 μg/ml) luc^{Fir}-dsRNA significantly suppressed luc^{Fir} activity. By contrast, control non-silencing siRNA, 100 bp and 500 bp luc^{Ren}-dsRNA had no effect.

FIG. 2B shows a soaking of SID-1-GFP-expressing HEK cells in 100 bp (30 and 100 μg/ml) or 500 bp (30 μg/ml) luc^{Fir}-dsRNA significantly suppressed luc^{Fir} activity. By contrast, control luc^{Ren}-dsRNA did not exert any luc^{Fir} suppressive effect.

FIG. 2C shows a soaking of SID-1-GFP-expressing HEK cells in luc^{Fir}-siRNA (30 and 50 μg/ml) significantly suppressed luc^{Fir} activity. By contrast, control non-silencing siRNA did not exert any luc^{Fir} suppressive effect. Data presented were averages from 3-5 experiments. *, p<0.05.

FIG. 3A is a representative image of GFP-expressing ESCs (left, phase-contrast; right, fluorescence).

FIG. 3B is a representative image of SID-1-GFP-expressing ESCs (left, phase-contrast; right, fluorescence).

FIG. 3C is a representative image of soaking of ESCs co-expressing SID-1-GFP+luc^{Fir} (but not GFP+luc^{Fir}) in 100 bp (30 and 100 μg/ml), 500 bp (30 μg/ml) luc^{Fir}-specific dsRNA led to suppression of luc^{Fir} activity. No suppression was observed for SID-1-GFP+luc^{Fir} and GFP+luc^{Fir} cells soaked with control luc^{Ren}-dsRNAs, indicating that RNAi was sequence-specific. Data presented were averages from 7-14 experiments. *, p<0.05.

FIG. 3D is a representative image of FACS analysis of GFP- or SID-1-GFP-expressing ESCs indicates that ectopic expression of SID-1 did not alter the percentage of early apoptotic (Annexin V-PE positive, 7-AAD negative) and dead cells (Annexin V-PE positive, 7-AAD positive).

### DETAILED DESCRIPTION OF THE INVENTION

As used herein, certain terms have the following defined meanings.

### DEFINITIONS

The practice of the present invention will employ, unless otherwise indicated, conventional techniques of immunology, molecular biology, microbiology, cell biology and recombinant DNA, which are within the skill of the art. See, e.g., Sambrook, Fritsch and Maniatis (1989) Molecular Cloning: A Laboratory Manual, 2^{nd} edition; Ausubel F. M., et al. eds., (1987) Current Protocols In Molecular Biology; the series Methods in Enzymology (Academic Press, Inc.); MacPherson, M. J., et al. eds. (1995) PCR 2: A Practical Approach; Harlow and Lane, eds. (1988) Antibodies, a Laboratory Manual, and Animal Cell Culture (R. I. Freshney, ed. (1987)).

As used in the specification and claims, the singular form “a”, “an” and “the” include plural references unless the context clearly dictates otherwise. For example, the term “a cell” includes a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention or process steps to produce a composition or achieve an intended result. Embodiments defined by each of these transition terms are within the scope of this invention.

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. It also is to be understood, although not always explicitly stated, that the reagents described herein are merely exemplary and that equivalents of such are known in the art.

The term “polypeptide” is used in its broadest sense to refer to a compound of two or more subunit amino acids, amino acid analogs, or peptidomimetics. The subunits may be linked by peptide bonds. In another embodiment, the subunit may be linked by other bonds, e.g., ester, ether, etc. As used herein the term “amino acid” refers to either natural and/or unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is commonly called a polypeptide or a protein.

The term “isolated” means separated from constituents, cellular and otherwise, in which the polynucleotide, peptide, polypeptide, protein, antibody, or fragments thereof, are normally associated with in nature. In one aspect of this invention, an isolated polynucleotide is separated from the 3′ and 5′ contiguous nucleotides with which it is normally associated with in its native or natural environment, e.g., on the chromosome. As is apparent to those of skill in the art, a non-naturally occurring polynucleotide, peptide, polypeptide, protein, antibody, or fragments thereof, does not require “isolation” to distinguish it from its naturally occurring counterpart. In addition, a “concentrated”, “separated” or “diluted” polynucleotide, peptide, polypeptide, protein, antibody, or fragments thereof, is distinguishable from its naturally occurring counterpart in that the concentration or number of molecules per volume is greater than “concentrated” or less than “separated” than that of its naturally occurring counterpart. A polynucleotide, peptide, polypeptide, protein, antibody, or fragments thereof, which differs from the naturally occurring counterpart in its primary sequence or for example, by its glycosylation pattern, need not be present in its isolated form since it is distinguishable from its naturally occurring counterpart by its primary sequence, or alternatively, by another characteristic such as glycosylation pattern. Thus, a non-naturally occurring polynucleotide is provided as a separate embodiment from the isolated naturally occurring polynucleotide. A protein produced in a bacterial cell is provided as a separate embodiment from the naturally occurring protein isolated from a eukaryotic cell in which it is produced in nature.

The terms “polynucleotide” and “oligonucleotide” are used interchangeably, and refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: a gene or gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

The term “polynucleotide sequence” is the alphabetical representation of a polynucleotide molecule. This alphabetical representation can be input into databases in a computer having a central processing unit and used for bioinformatics applications such as functional genomics and homology searching.

A “gene” refers to a polynucleotide containing at least one open reading frame that is capable of encoding a particular polypeptide or protein after being transcribed and translated. Any of the polynucleotides sequences described herein may be used to identify larger fragments or full-length coding sequences of the gene with which they are associated. Methods of isolating larger fragment sequences are known to those of skill in the art, some of which are described herein. An “exogenous gene” refers to a gene introduced into the cell, and may be either a non-native sequence or may be the native sequence of the gene if that gene also naturally occurs within the cell.

A “gene product” or “expression product” refers to the amino acid (e.g., peptide or polypeptide) generated when a gene is transcribed and translated.

“Under transcriptional control” is a term well understood in the art and indicates that transcription of a polynucleotide sequence, usually a DNA sequence, depends on its being operatively linked to an element which contributes to the initiation of, or promotes, transcription. “Operatively linked” intends the polynucleotides are arranged in a manner that allows them to function in a cell.

A “gene delivery vehicle” is defined as any molecule that can carry inserted polynucleotides into a host cell. Examples of gene delivery vehicles are liposomes, biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

“Gene delivery,” “gene transfer,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection, sometimes called transduction), transfection, transformation or various other protein-based or lipid-based gene delivery complexes) as well as techniques facilitating the delivery of “naked” polynucleotides (such as electroporation, “gene gun” delivery and various other techniques used for the introduction of polynucleotides). Unless otherwise specified, the term transfected, transduced or transformed may be used interchangeably herein to indicate the presence of exogenous polynucleotides or the expressed polypeptide therefrom in a cell. The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in an eukaryotic cell. “Differentially expressed” as applied to a gene, refers to the differential production of the mRNA transcribed from the gene or the protein product encoded by the gene. A differentially expressed gene may be overexpressed or underexpressed as compared to the expression level of a normal or control cell. In one aspect, it refers to overexpression that is 1.5 times, or alternatively, 2 times, or alternatively, at least 2.5 times, or alternatively, at least 3.0 times, or alternatively, at least 3.5 times, or alternatively, at least 4.0 times, or alternatively, at least 5 times, or alternatively at least 10 times or more higher (and therefore overexpressed) or lower than the expression level detected in a control sample. The term “differentially expressed” also refers to nucleotide sequences in a cell or tissue which are expressed where silent in a control cell or not expressed where expressed in a control cell. The term “stable expression” is defined infra.

A cell that “stably expresses” an exogenous polypeptide is one that continues to express a polypeptide encoded by an exogenous gene introduced into the cell either after replication if the cell is dividing or for longer than a day, up to about a week, up to about two weeks, up to three weeks, up to four weeks, for several weeks, up to a month, up to two months, up to three months, for several months, up to a year or more.

A “viral vector” is defined as a recombinantly produced virus or viral particle that comprises a polynucleotide to be delivered into a host cell, either in vivo, ex vivo or in vitro. Examples of viral vectors include retroviral vectors, lentiviral vectors, adenovirus vectors, adeno-associated virus vectors, alphavirus vectors and the like. Alphavirus vectors, such as Semliki Forest virus-based vectors and Sindbis virus-based vectors, have also been developed for use in gene therapy and immunotherapy. See, Schlesinger and Dubensky (1999) Curr. Opin. Biotechnol. 5:434-439 and Ying, et al. (1999) Nat. Med. 5(7):823-827.

In aspects where gene transfer is mediated by a retroviral vector, a vector construct refers to the polynucleotide comprising the retroviral genome or part thereof, and a therapeutic gene. As used herein, “retroviral mediated gene transfer” or “retroviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. Retroviruses carry their genetic information in the form of RNA; however, once the virus infects a cell, the RNA is reverse-transcribed into the DNA form which integrates into the genomic DNA of the infected cell. The integrated DNA form is called a provirus. As used herein, retroviral vector refers to a viral particle capable of introducing exogenous nucleic acid into a cell through a viral or viral-like entry mechanism. A “lentiviral vector” is a type of retroviral vector well-known in the art that has certain advantages in transducing nondividing cells as compared to other retroviral vectors. See, Trono D. (2002) Lentiviral vectors, New York: Spring-Verlag Berlin Heidelberg.

In aspects where gene transfer is mediated by a DNA viral vector, such as an adenovirus (Ad) or adeno-associated virus (AAV), a vector construct refers to the polynucleotide comprising the viral genome or part thereof, and a transgene. Adenoviruses (Ads) are a relatively well characterized, homogenous group of viruses, including over 50 serotypes. See, e.g., International PCT Application No. WO 95/27071. Ads do not require integration into the host cell genome. Recombinant Ad derived vectors, particularly those that reduce the potential for recombination and generation of wild-type virus, have also been constructed. See, International PCT Application Nos. WO 95/00655 and WO 95/11984. Wild-type AAV has high infectivity and specificity integrating into the host cell's genome. See, Hermonat and Muzyczka (1984) Proc. Natl. Acad. Sci. USA 81:6466-6470 and Lebkowski, et al. (1988) Mol. Cell. Biol. 8:3988-3996.

Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

Gene delivery vehicles also include several non-viral vectors, including DNA/liposome complexes, and targeted viral protein-DNA complexes. Liposomes that also comprise a targeting antibody or fragment thereof can be used in the methods of this invention. To enhance delivery to a cell, the nucleic acid or proteins of this invention can be conjugated to antibodies or binding fragments thereof which bind cell surface antigens, e.g., a cell surface marker found on stem cells.

A “probe” when used in the context of polynucleotide manipulation refers to an oligonucleotide that is provided as a reagent to detect a target potentially present in a sample of interest by hybridizing with the target. Usually, a probe will comprise a label or a means by which a label can be attached, either before or subsequent to the hybridization reaction. Suitable labels are described and exemplified herein.

A “primer” is a short polynucleotide, generally with a free 3′-OH group that binds to a target or “template” potentially present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or a “set of primers” consisting of an “upstream” and a “downstream” primer, and a catalyst of polymerization, such as a DNA polymerase, and typically a thermally-stable polymerase enzyme. Methods for PCR are well known in the art, and taught, for example in M. MacPherson et al. (1991) PCR: A Practical Approach, IRL Press at Oxford University Press. All processes of producing replicate copies of a polynucleotide, such as PCR or gene cloning, are collectively referred to herein as “replication.” A primer can also be used as a probe in hybridization reactions, such as Southern or Northern blot analyses. Sambrook et al., supra. The primers may optionally contain detectable labels and are exemplified and described herein.

As used herein, the term “label” intends a directly or indirectly detectable compound or composition that is conjugated directly or indirectly to the composition to be detected, e.g., polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g. radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluoresecence assays, the detectable response may be generated directly using a luminophore or fluorophore associated with an assay component actually involved in binding, or indirectly using a luminophore or fluorophore associated with another (e.g., reporter or indicator) component.

Examples of luminescent labels that produce signals include, but are not limited to bioluminescence and chemiluminescence. Detectable luminescence response generally comprises a change in, or an occurrence of, a luminescence signal. Suitable methods and luminophores for luminescently labeling assay components are known in the art and described for example in Haugland, Richard P. (1996) Handbook of Fluorescent Probes and Research Chemicals (6^{th} ed.). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6^{th} ed.).

In another aspect, the fluorescent label is functionalized to facilitate covalent attachment to a cellular component present in or on the surface of the cell or tissue such as a cell surface marker. Suitable functional groups, including, but not are limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

Attachment of the fluorescent label may be either directly to the cellular component or compound or alternatively, can by via a linker. Suitable binding pairs for use in indirectly linking the fluorescent label to the intermediate include, but are not limited to, antigens/antibodies, e.g., rhodamine/anti-rhodamine, biotin/avidin and biotin/strepavidin.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Hybridization reactions can be performed under conditions of different “stringency”. In general, a low stringency hybridization reaction is carried out at about 40° C. in 10×SSC or a solution of equivalent ionic strength/temperature. A moderate stringency hybridization is typically performed at about 50° C. in 6×SSC, and a high stringency hybridization reaction is generally performed at about 60° C. in 1×SSC.

When hybridization occurs in an antiparallel configuration between two single-stranded polynucleotides, the reaction is called “annealing” and those polynucleotides are described as “complementary”. A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to form hydrogen bonding with each other, according to generally accepted base-pairing rules.

A polynucleotide or polynucleotide region (or a polypeptide or polypeptide region) has a certain percentage (for example, 80%, 85%, 90%, or 95%) of “sequence identity” to another sequence means that, when aligned, that percentage of bases (or amino acids) are the same in comparing the two sequences. This alignment and the percent homology or sequence identity can be determined using software programs known in the art, for example those described in F. M. Ausubel et al., eds. (1987) Current Protocols in Molecular Biology Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: http://www.ncbi.nlm.nih.gov/cgi-bin/BLAST.

As used herein, “stem cell” defines a cell with the ability to divide for indefinite periods in culture and give rise to specialized cells. At this time and for convenience, stem cells are categorized as somatic (adult) or embryonic. A somatic stem cell is an undifferentiated cell found in a differentiated tissue that can renew itself (clonal) and (with certain limitations) differentiate to yield all the specialized cell types of the tissue from which it originated. An embryonic stem cell is a primitive (undifferentiated) cell from the embryo that has the potential to become a wide variety of specialized cell types. An embryonic stem cell is one that has been cultured under in vitro conditions that allow proliferation without differentiation for months to years. A clone is a line of cells that is genetically identical to the originating cell; in this case, a stem cell.

“Clonal proliferation” refers to the growth of a population of cells by the continuous division of single cells into two identical daughter cells.

As used herein, a “pluripotent cell” defines a less differentiated cell that can give rise to at least two distinct (genotypically and/or phenotypically) further differentiated progeny cells.

A “multi-lineage stem cell” or “multipotent stem cell” refers to a stem cell that reproduces itself and at least two further differentiated progeny cells from distinct developmental lineages. The lineages can be from the same germ layer (i.e. mesoderm, ectoderm or endoderm), or from different germ layers. An example of two progeny cells with distinct developmental lineages from differentiation of a multilineage stem cell is a myogenic cell and an adipogenic cell (both are of mesodermal origin, yet give rise to different tissues). Another example is a neurogenic cell (of ectodermal origin) and adipogenic cell (of mesodermal origin).

“Differentiation” describes the process whereby an unspecialized cell acquires the features of a specialized cell such as a heart, liver, or muscle cell. “Directed differentiation” refers to the manipulation of stem cell culture conditions to induce differentiation into a particular cell type. “Dedifferentiated” defines a cell that reverts to a less committed position within the lineage of a cell. As used herein, the term “differentiates or differentiated” defines a cell that takes on a more committed (“differentiated”) position within the lineage of a cell. As used herein, “a cell that differentiates into a mesodermal (or ectodermal or endodermal) lineage” defines a cell that becomes committed to a specific mesodermal, ectodermal or endodermal lineage, respectively. Examples of cells that differentiate into a mesodermal lineage or give rise to specific mesodermal cells include, but are not limited to, cells that are adipogenic, leiomyogenic, chondrogenic, cardiogenic, dermatogenic, hematopoetic, hemangiogenic, myogenic, nephrogenic, urogenitogenic, osteogenic, pericardiogenic, or stromal.

Examples of cells that differentiate into ectodermal lineage include, but are not limited to epidermal cells, neurogenic cells, and neurogliagenic cells.

Examples of cells that differentiate into endodermal lineage include, but are not limited to pleurogenic cells, and hepatogenic cells, cell that give rise to the lining of the intestine, and cells that give rise to pancreogenic and splanchogenic cells.

As used herein, an “antibody” includes whole antibodies and any antigen binding fragment or a single chain thereof. Thus the term “antibody” includes any protein or peptide containing molecule that comprises at least a portion of an immunoglobulin molecule. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein, any of which can be incorporated into an antibody of the present invention. The term “antibody” is further intended to encompass digestion fragments, specified portions, derivatives and variants thereof, including antibody mimetics or comprising portions of antibodies that mimic the structure and/or function of an antibody or specified fragment or portion thereof, including single chain antibodies and fragments thereof. Examples of binding fragments encompassed within the term “antigen binding portion” of an antibody include a Fab fragment, a monovalent fragment consisting of the V_{L}, V_{H}, C_{L} and C_{H}, domains; a F(ab′)2 fragment, a bivalent fragment comprising two Fab fragments linked by a disulfide bridge at the hinge region; a Fd fragment consisting of the V_{H} and C_{H}, domains; a Fv fragment consisting of the V_{L} and V_{H} domains of a single arm of an antibody, a dAb fragment (Ward et al. (1989) Nature 341:544-546), which consists of a V_{H} domain; and an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv)). Bird et al. (1988) Science 242:423-426 and Huston et al. (1988) Proc. Natl. Acad. Sci. USA 85:5879-5883. Single chain antibodies are also intended to be encompassed within the term “fragment of an antibody.” Any of the above-noted antibody fragments are obtained using conventional techniques known to those of skill in the art, and the fragments are screened for binding specificity and neutralization activity in the same manner as are intact antibodies.

“RNA interference” (RNAi) refers to sequence-specific or gene specific suppression of gene expression (protein synthesis) that is mediated by short interfering RNA (siRNA).

“Short interfering RNA” (siRNA) refers to double-stranded RNA molecules (dsRNA), generally, from about 10 to about 30 nucleotides in length that are capable of mediating RNA interference (RNAi), or 11 nucleotides in length, 12 nucleotides in length, 13 nucleotides in length, 14 nucleotides in length, 15 nucleotides in length, 16 nucleotides in length, 17 nucleotides in length, 18 nucleotides in length, 19 nucleotides in length, 20 nucleotides in length, 21 nucleotides in length, 22 nucleotides in length, 23 nucleotides in length, 24 nucleotides in length, 25 nucleotides in length, 26 nucleotides in length, 27 nucleotides in length, 28 nucleotides in length, or 29 nucleotides in length. As used herein, the term siRNA includes short hairpin RNAs (shRNAs).

“Double stranded RNA” (dsRNA) refer to double stranded RNA molecules that may be of any length and may be cleaved intracellularly into smaller RNA molecules, such as siRNA. In cells that have a competent interferon response, longer dsRNA, such as those longer than about 30 base pair in length, may trigger the interferon response. In other cells that do not have a competent interferon response, dsRNA may be used to trigger specific RNAi.

A “composition” is intended to mean a combination of active agent and another compound or composition, inert (for example, a detectable agent or label) or active, such as an adjuvant.

A “pharmaceutical composition” is intended to include the combination of an active agent with a carrier, inert or active, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

An “effective amount” is an amount sufficient to effect beneficial or desired results. An effective amount can be administered in one or more administrations, applications or dosages.

### EMBODIMENTS

This invention provides a eukaryotic cell transformed with exogenous SID-1 polynucleotide, wherein the cell stably expresses or overexpresses the exogenous SID-1 polynucleotide to confer siRNA or dsRNA uptake in the transfected or transduced cells when exposed to the siRNA or dsRNA, such as by soaking the cell in a solution comprising the siRNA or dsRNA. In one aspect, the eukaryotic cell is an isolated animal cell, examples of which include but are not limited to a mammalian cell e.g., a bovine, a murine cell, a simian cell, a porcine cell or a human cell. In a particular aspect, the animal cell is a cultured human embryonic kidney cell (HEK 293T). In an alternative embodiment, the animal cell is an isolated stem cell, e.g., somatic or isolated embryonic stem cell. The stem cell can be of human or animal origin. The transformed cells are particularly useful for high throughput screening of gene activity, e.g, via RNAi. Methods for isolation, culturing and differentiation of eukaryotic cells, e.g., stem cells, are known in the art and briefly described herein.

For the purpose of this invention, the SID-1 polynucleotide is DNA polynucleotide or RNA polynucleotide. “SID-1” intends a polynucleotide that was originally identified in C. elegans (Swiss Prot Accession No. Q9GZC8, Genbank Accession No. NM_{—}071971). Members of the family and homologs can be found at Genbank Accession Nos. NM_{—}017699 (human), NP_{—}060169 (human) and AAI17223 (human), Swiss Prot Accession No. Q6Q3F5 (rat), Swiss Prot Accession No. Q6Q3F6 (mouse). SID-1 polynucleotides also intends polynucleotides that encode SID-1 polypeptides, e.g., the sequences of which are disclosed herein and disclosed in GenBank Accession Nos. NM_{—}017699 (human), NM_{—}017699 (human), AAI17223 (human) and AF478687 (C. elegans). In one aspect, the SID-1 polynucleotide is any one of these family members, including polynucleotides and proteins having greater than 90%, or alternatively, greater than 95%, or alternatively, greater than 98% sequence homology, using the parameters described herein when run under default conditions. In a particular embodiment, the SID-1 polynucleotide is exogenous to the eukaryotic cell. In an alternate embodiment, the SID-1 polynucleotide is endogenous but overexpression of the gene product is achieve by insertion of an enhancing element upstream to the gene for SID-1 polypeptide. This technology is described in the art, e.g., Xue, et al (2005) Circulation 111:11-20.

In yet another aspect of this invention, the eukarytic cell, such as a mammalian stem cell, further comprises an RNA polynucleotide. In a particular embodiment, the RNA polynucleotide is siRNA. In an alternative embodiment, the RNA polynucleotide is dsRNA. The RNA polynucleotide may further comprise a detectable label, for example, but not limited to, a fluorescent label which is detectable by flow cytometry, Western blot, or MACS® (Miltenyi Biotec, California) methods.

In another aspect, the RNA polynucleotide is siRNA or dsRNA that is complementary to a gene having biological activity selected from the group consisting of apoptosis, cell adhesion, cell cycle, immunotherapy, cell signaling, DNA repair, DNA synthesis. In one embodiment, polynucleotide is complementary to a gene having biological activity in immunotherapy, e.g., a cytokine, chemokine, transcriptional regulation factor or a translational regulation factor.

Also provided by this invention is a method for preparing a eukaryotic cell, such as a mammalian stem cell, with an exogenous siRNA or dsRNA polynucleotide, by passively contacting a stably transformed eukaryotic cell overexpressing exogenous SID-1 polypeptide with the exogenous siRNA or dsRNA. As used herein, the term “passively contacting” means contacting the cell with the siRNA or dsRNA, such as by soaking, in the absence of a vector or other means typically used by those of skill in the art to facilitate insertion of polynucleotides, e.g. siRNA or dsRNA, into eukaryotic cells.

In one aspect, the eukaryotic cell is an isolated animal cell, examples of which include but are not limited to a mammalian cell, a murine cell, a simian cell, a bovine cell, a porcine cell or a human cell. In a particular aspect, the animal cell is a cultured human embryonic kidney cell (HEK 293T). In an alternative embodiment, the animal cell is an isolated stem cell, e.g., somatic or isolated embryonic stem cell. The stem cell can be of human or animal origin, such as mammalian. The transformed cells are particularly useful for high throughput screening of gene activity through the use of RNAi. Methods for isolation, culturing and differentiation of eukaryotic cells, e.g., stem cells are known in the art and briefly described herein.

This invention provides a stably transfected or transduced mammalian stem cell that expresses exogenous SID-1 to confer enhanced siRNA or dsRNA uptake in the cells. Thus, in one aspect, this invention provides a mammalian stem cell transfected or transduced with exogenous SID-1 polynucleotide, wherein the cell stably expresses or overexpresses the exogenous SID-1 polynucleotide and is capable of take up siRNA or dsRNA through passive contact or soaking. In one embodiment, the mammalian stem cell is a mouse embryonic stem cell. In an alternative embodiment, the mammalian stem cell is a human stem cell. The human stem cell can be a somatic stem cell or alternatively an embryonic stem cell, and the stem cell may be differentiated or pluripotent. The mammalian stem cell may be identified by whether it expresses a particular marker. For example, a mouse embryonic stem cell may be identified by whether it expresses a marker such as, but not limited to, SSEA-1. Also for example, a human embryonic stem cell may be identified by whether it expresses a marker such as, but not limited to, SSEA-3/4, TRA-1-81, or TRA-1-60. Markers for identifying mammalian stem cells are readily commercially available and procedures for using these markers to identify mammalian stem cells are well known to persons of ordinary skill in the art.

It should be understood, although not always explicitly stated, that an effective amount of the polynucleotides described herein are introduced into the eukaryotic cell.

After introduction of the polynucleotide into the eukaryotic cell, e.g., mammalian cell, the cell can be propagated to produce a population of transduced cells. In aspect where the transduced cell is a stem cell, the stem cell can be propagated under conditions to produce a clonal population or alternatively, a differentiated cell population, each stably expressing SID-1. The cell or cell may be further combined with a carrier such as a pharmaceutically acceptable carrier.

For the purpose of this method, the SID-1 polynucleotide is DNA polynucleotide or RNA polynucleotide. “SID-1” intends a polynucleotide that was originally identified in C. elegans. (Swiss Prot Accession No. Q9GZC8, Genbank Accession No. NM_{—}071971 and GenBank Accession No. AF478687). Members of the family and homologs can be found at Genbank Accession Nos. NM_{—}017699 (human), NP AK000181 (human) and AAI17223 (human), Swiss Prot Accession No. Q6Q3F5 (rat), Swiss Prot Accession No. Q6Q3F6 (mouse). SID-1 polynucleotides also intends polynucleotides that encode SID-1 polypeptides, e.g., the sequences of which are disclosed herein and disclosed in GenBank Accession Nos. NM_{—}017699 (human), NM_{—}017699 (human), AAI17223 (human) and AF478687 (C. elegans). In one aspect, the SID-1 polynucleotide is any one of these family members, including polynucleotides and proteins having greater than 90%, or alternatively, greater than 95%, greater than 98% or alternatively, greater than 99% sequence homology, using the parameters described herein when run under default conditions. In another aspect, the SID-1 polynucleotide can encode a polypeptide that exhibits SID-1 function in facilitating the uptake of polynucleotides. In one embodiment, the encoded polypeptide is a SID-1 related protein, its homolog or a variant thereof.

In a particular embodiment, the SID-1 polynucleotide is exogenous to the eukaryotic cell. In an alternate embodiment, the SID-1 polynucleotide is endogenous but overexpression of the gene product is achieve by insertion of an enhancing element upstream to the gene for SID-1 polypeptide. This technology is described in the art, e.g., Xue, et al (2005) Circulation 111:11-20.

In another aspect, the RNA polynucleotide that is used in this method is siRNA or dsRNA that is complementary to a gene having biological activity selected from the group consisting of apoptosis, cell adhesion, cell cycle, immunotherapy, cell signaling, DNA repair, DNA synthesis. In one embodiment, the polynucleotide that is used is complementary to a gene having biological activity in immunotherapy, e.g., a cytokine, chemokine, transcriptional regulation factor or a translational regulation factor.

Also provided by this invention is a method for preparing a mammalian stem cell with an exogenous siRNA or dsRNA polynucleotide, by passively contacting a stably transformed mammalian stem cell overexpressing exogenous SID-1 polypeptide with the exogenous siRNA or dsRNA. As used herein, the term “passively contacting”, also called “soaking” means contacting the cell with the siRNA or dsRNA, in the absence of a vector, transfection reagent or other means typically used by those of skill in the art to facilitate insertion of exogenous genes or polynucleotides into eukaryotic cells.

In a particular aspect, the eukaryotic cell is co-transformed with siRNA or dsRNA. In a further aspect, this RNA can be detectably labeled with, for example, a fluorescent label which is detectable by FACS or MACS methods.

The eukaryotic cells can be used in a method for detecting gene specific interference by an exogenous siRNA or dsRNA and resultant functional consequences by comparing the change in label detected from a cell as described above at least two distinct time points, such as before and after addition of the exogenous siRNA or dsRNA. The time points can be at least 15 minutes, or alternatively at least 30 minutes, or alternatively at least 1 hour, or alternatively at least 2 hours, or alternatively at least 4 hours, or alternatively, at least 6 hours, or alternatively, at least 12 hours, or alternatively, at least 1 day, apart.

Fluorescence activated cell sorting (FACS), also called flow cytometry, is used to sort individual cells on the basis of optical properties, including fluorescence. It is used to screen large populations of cells in a relatively short period of time. Other screening methods include MACs, as described in Gaines (1999) Biotechniques 26(4):683-688 and Western blot as described herein.

Suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in Haugland, Richard P. (1996) Molecular Probes Handbook.

A “high throughput screen” is assaying more than one gene activity via RNAi without the need for generating multiple viral constructs. Cells, such as stem cells, stably expressing SID-1 can be contacted with, such as by soaking, more than one polynucleotide, for example, more than one siRNA or dsRNA, in parallel. By avoiding the need for multiple transfections or transductions with gene delivery vehicles, assaying the activity of more than one gene is greatly simplified and more efficient.

### Eukaryotic Cells

Examples of suitable eukaryotic cells include, but are not limited to, the exemplified 293T HEK cells, as well as the hamster cell line BHK-21; the murine cell lines designated NIH3T3, NS0, C127, the simian cell lines COS, Vero; and the human cell lines HeLa, PER.C6 (commercially available from Crucell) U-937 and Hep G2. Additional examples include yeast cells, insect cells, plant cells, avian cells, fungal cells and bovine cells. Examples of yeast useful for expression include, but are not limited to Saccharomyces, Schizosaccharomyces, Hansenula, Candida, Torulopsis, Yarrowia, or Pichia. See e.g., U.S. Pat. Nos. 4,812,405; 4,818,700; 4,929,555; 5,736,383; 5,955,349; 5,888,768 and 6,258,559. The eukaryotic cells can be purchased from a commercial vendor such as the American Type Culture Collection (ATCC, Rockville Md., USA) or cultured from an isolate using methods known in the art.

In one aspect, the host cells have been pre-screened and selected for stable production SID-1 polypeptide. Chemical selection with methotrexate is one method for achieving and selecting stable transformants, but other selection methods are known in the art with some examples described in Liu et al. (2000) Anal. Biochem. 280:20-28; Barnes et al. (2003) Biotechnol. Bioeng. 81:631-639; Sautter and Enenkel (2005) Biotechnol. Bioeng. 89:530-538. In an alternate embodiment, the cells have not been pre-selected for stable production of SID-1.

### Stem Cells

Presently, somatic stem cells are described by the tissue from which they are isolated and in some cases, further by certain cell surface markers. Stem cells have been isolated from hemaotpoeitic tissue (hematopoietic stem cells or “HSCs”), mesenchymal tissue (mesenchymal stem cells or “MSC”), the heart (cardiac resident stem cells), the brain and central nervous system (neural progenitor cells) and adipose tissue (Adipose-derived stem cells or “ADSCs”), for example. See Verfaillie, C. (2004) “Adult Stem Cells: Tissue Specific or Not ?”, Handbook of Stem Cells, Vol. 2. Any of these stem cells types or others known in the art can be isolated and transduced with polynucleotides. The somatic stem cells can be isolated, cultured, differentiated or clonally expanded, as determined by those of skill in the art.

In a separate embodiment, the stem cell is an embryonic stem cell. Embryonic stem cells are derived from embryos that develop from eggs that have been fertilized in vitro and then donated for research purposes. There is still dispute in the scientific community over the identifying characteristics of embryonic stem cells but the following are some of the characteristics published by the National Institutes of Health (NIH) on their web site which can be accessed at the web address “stemcells.nih.gov/info/basics/basics3.asp” (last accessed on Jan. 25, 2006):

1) the presence of a protein called Oct-4, which undifferentiated cells typically make. Oct-4 is a transcription factor;
2) the phenotype, when examined microscopically should show that the chromosomes are unchanged and undamaged; and
3) evidence of pluripotentency by 1) allowing the cells to differentiate spontaneously in cell culture; 2) manipulating the cells so they will differentiate to form specific cell types; or 3) injecting the cells into an immunosuppressed mouse to test for the formation of a benign tumor called a teratoma. Teratomas typically contain a mixture of many differentiated or partly differentiated cell types—an indication that the embryonic stem cells are capable of differentiating into multiple cell types.

Methods to isolate embryonic stem cells have been reported in the patent and technical literature. For example, U.S. Pat. No. 6,280,718, issued Aug. 28, 2001 discloses methods of obtaining human pluripotent embryonic stem cells using mammalian stromal cells. See also U.S. Pat. No. 6,200,806, issued Mar. 13, 2001. U.S. Patent Appl. No.: 20050260747, published Nov. 24, 2005 discloses methods to obtain and culture undifferentiated human embryonic stem cells as well as methods and compositions to differentiate the cells in vitro. U.S. Patent Appl. No.: 20060015961, published Jan. 19, 2006, discloses the isolation and use of peripheral blood derived germ stem cells. Embryonic stem cell lines are also available through WiCell Research Institute, Madison Wis., which can be accessed through the web site at the address “wicell.org” (last accessed on Jan. 25, 2006).

Stem cells for use in this invention can be obtained from any animal (alive or dead) for use in the invention and animal model (described infra) so long as stem cells within the animal are viable.

The use of stem cells stably expressing SID-1 enables screening of the activity and identification of genes important for development via RNAi. Typical cell lines and primary cell types have their fates committed and are terminally differentiated. Stem cells, on the other hand, are not terminally differentiated and remain multipotent or totipotent. Thus, a potentially broader range of genes are expressed, enabling study of their activity by RNAi by the methods presented herein. Furthermore, a stem cell stably expressing SID-1 may provide a population of cells expressing SID-1 for further study of gene activity by RNAi.

Stem cells stably expressing SID-1 may be used for high throughput screening of gene activity through the use of RNAi, as cells can be contacted with or soaked in a solution containing siRNA or dsRNA polynucleotides as a fast method for assaying gene function. Traditional gene activity studies require tedious knockout methods or the development of gene delivery vehicles and subsequent transfections or transductions. Furthermore, the lack of an interferon response in stem cells may facilitate specific RNAi induction, especially by dsRNA longer than about 30 base pair in length. Thus, stem cells stably expressing SID-1 represent a useful tool for studying gene activity.

### Transduction with Carriers or Vectors

Exogenous SID-1 polynucleotides can be inserted into the eukaryotic cell by use of any appropriate gene delivery vehicle. In one aspect, the gene delivery vehicle is a vector. As known to those skilled in the art, appropriate regulatory sequences may need to be operatively linked to enhance or insure expression of the polynucleotide in the host cell.

Suitable vectors include, but are not limited to viral vectors, viral-associated vectors and plasmids. Examples of suitable DNA viral vectors include lentivirus as described herein as well as adenovirus (Ad) or adeno-associated virus (AAV). Adenovirus-based vectors for the delivery of polynucleotides are known in the art and may be obtained commercially or constructed by standard molecular biological methods. Adenoviruses (Ads) are a group of viruses, including over 50 serotypes. See, e.g., International PCT Application No. WO 95/27071. Adenoviruses do not require integration into the host cell genome. Recombinant Ad derived vectors, particularly those that reduce the potential for recombination and generation of wild-type virus, have also been constructed. See, International PCT Application Nos. WO 95/00655 and WO 95/11984. In general, recombinant adenoviral vectors derived from adenovirus type 2 (Ad2) and adenovirus type 5 (Ad5). They may also be derived from other non-oncogenic serotypes. See, for example, Horowitz (1990) “Adenoviridae and their Replication” in Virology, 2d ed., Fields et al. eds., Raven Press Ltd., New York.

Other viral vectors for use in the present invention include vectors derived from lentivirus, vaccinia, herpesvirus, and retroviruses, which have been discussed previously.

Vectors that contain both a promoter and a cloning site into which a polynucleotide can be operatively linked are known in the art and available from commercial vendors. Such vectors are capable of transcribing RNA in vitro or in vivo, and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

Gene delivery vehicles also include several non-viral vectors, including DNA/liposome complexes, and targeted viral protein-DNA complexes. Liposomes that also comprise a targeting antibody or fragment thereof can be used in the methods of this invention.

After insertion of SID-1 polynucleotide, the host cell is cultured under conditions that facilitate expression of the polynucleotide. Any method known in the art useful for detecting the SID-1 expressed on the cell surface may be used in connection with the methods of the invention. For example, an antibody or other cell surface marker-specific binding agent is then contacted directly or indirectly with the cell under conditions that favor binding of antibody to the marker and therefore the host cell.

### Design of siRNAs

siRNA sequences are designed, for example, by obtaining the target mRNA sequence and determining an appropriate siRNA complementary sequence. siRNAs of the invention are designed to interact with a target sequence, meaning they complement a target sequence sufficiently to anneal with that sequence.

The siRNA can be 100% identical to the target sequence, or complement thereof. However, homology of the siRNA sequence to the target sequence can be less than 100% as long as the siRNA is capable of annealing with the target sequence. Thus, for example, the siRNA molecule can be at least 80%, or alternatively, at least 85%, or alternatively, at least 90%, or alternatively, at least 95%, or alternatively, at least 96%, or alternatively, at least 97%, or alternatively, at least 98%, or alternatively, at least 99%, or alternatively, 100% identical to the target sequence or the complement of the target sequence. Therefore, siRNA molecules with insertions, deletions or single point mutations relative to a target sequence may also be used. The generation of several different siRNA sequences per target mRNA is recommended to allow screening for the optimal target sequence. A homology search, such as a BLAST search, should be performed to ensure that the siRNA sequence does not contain homology to any known mammalian gene.

In general, its preferable that the target sequence be located at least 100-200 nucleotides from the AUG initiation codon and at least 50-100 nucleotides away from the termination codon of the target mRNA Duxbury M. S. et al. (2005) Biochem Biophys Res Commun 331:459-463.

Researchers have determined that certain characteristics are common in siRNA molecules that effectively silence their target gene. Duxbury M. S. et al. (2005) Biochem Biophys Res Commun 331:459-463; Ui-Tei, K. et al. (2004) Nucl. Acids Res. 32:936-48. As a general guide, siRNAs that include one or more of the following conditions are particularly useful in gene silencing in mammalian cells:GC ratio of between 45-55%, no runs of more than 9 G/C residues, G/C at the 5′ end of the sense strand; A/U at the 5′ end of the antisense strand; and at least 5 A/U residues in the first 7 bases of the 5′ terminal of the antisense strand.

siRNA are, in general, from about 10 to about 30 nucleotides in length. For example, the siRNA can be 10-30 nucleotides in length, or alternatively, 12-28 nucleotides in length, or alternatively, 15-25 nucleotides in length, or alternatively, 19-23 nucleotides in length, or alternatively, 21-23 nucleotides in length. When an siRNA contains two strands of different lengths, the longer of the strands designates the length of the siRNA. In this situation, the unpaired nucleotides of the longer strand would form an overhang.

The term siRNA includes short hairpin RNAs (shRNAs). shRNAs comprise a single strand of RNA that forms a stem-loop structure, where the stem consists of the complementary sense and antisense strands that comprise a double-stranded siRNA, and the loop is a linker of varying size. The stem structure of shRNAs generally is from about 10 to about 30 nucleotides in length. For example, the stem can be 10-30 nucleotides in length, or alternatively, 12-28 nucleotides in length, or alternatively, 15-25 nucleotides in length, or alternatively, 19-23 nucleotides in length, or alternatively, 21-23 nucleotides in length.

Tools to assist siRNA design are readily available to the public. For example, a computer-based siRNA design tool is available on the internet at www.dharmacon.com.

### Synthesis of dsRNA and siRNA

dsRNA and siRNA can be synthesized chemically or enzymatically in vitro. Micura, R. (2002) Agnes Chem. Int. Ed. Emgl 41: 2265-9; Betz, N. (2003) Promega Notes 85:15-18; Paddison, P. J. and Hannon, G. J. (2002) Cancer Cell. 2:17-23. Chemical synthesis can be performed via manual or automated methods, both of which are well known in the art. Micura, R. (2002) Agnes Chem. Int. Ed. Emgl. 41: 2265-9. siRNA can also be endogenously expressed inside the cells in the form of shRNAs. Yu, J-Y. et al. (2002) Proc. Natl. Acad. Sci. USA 99: 6047-52; McManus, M. T. et al. (2002) RNA 8:842-50. Endogenous expression has been achieved using plasmid-based expression systems using small nuclear RNA promoters, such as RNA polymerase III U6 or H1, or RNA polymerase II U1. Brummelkamp, T. R. et al. (2002) Science 296:550-3; Novarino, G. et al. (2004) J. Neurosci. 24:5322-30.

In vitro enzymatic dsRNA and siRNA synthesis can be performed using an RNA polymerase mediated process to produce individual sense and antisense strands that are annealed in vitro prior to delivery into the cells of choice. Fire A. et al. (1998) Nature 391:806-811; Donze, O. and Picard, D. (2002) Nucl. Acids Res. 30 (10):e46; Yu, J-Y. et al. (2002) Proc. Natl. Acad. Sci. USA 99: 6047-52; Shim, E. Y. et al. (2002) J. Biol. Chem. 277:30413-6. Several manufacturers (Promega Corp. (Madison, Wis.); Ambion, Inc. (Austin, Tex.); New England Biolabs (Ipswich, Mass.); and Stragene (La Jolla, Calif.) provide transcription kits useful in performing the in vitro synthesis.

In vitro synthesis of siRNA can be achieved, for example, by using a pair of short, duplex oligonucleotides that contain T7 RNA polymerase promoters upstream of the sense and antisense RNA sequences as the DNA template. Each oligonucleotide of the duplex is a separate template for the synthesis of one strand of the siRNA. The separate short RNA strands that are synthesized are then annealed to form siRNA. Protocols and Applications, Chapter 2: RNA interference, Promega Corporation, (2005).

In vitro synthesis of dsRNA can be achieved, for example, by using a T7 RNA polymerase promoter operatively linked at the 5′-ends of both DNA target sequence strands. This is accomplished by using separate DNA templates, each containing the target sequence in a different orientation relative to the T7 promoter, transcribed in two separate reactions. The resulting transcripts are mixed and annealed post-transcriptionally. DNA templates used in this reaction can be created by PCR or by using two linearized plasmid templates, each containing the T7 polymerase promoter at a different end of the target sequence. Protocols and Applications, Chapter 2: RNA interference, Promega Corporation, (2005).

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

### EXAMPLES

### Cell Culture

Human embryonic kidney 293T (HEK) cells were cultured in Dulbecco's modified Eagle's medium (DMEM) (Gibco BRL; Carlsbad, Calif.) supplemented with 10% fetal bovine serum (Gibco BRL), 2 mM L-glutamine, 0.1 mM nonessential amino acids and 500 μg/mL geneticin (Invitrogen; Carlsbad, Calif.).

For mESCs, the ES-D3 line (ATCC; Manassas, Va.) was used. Pluripotency was maintained by growing mESCs on irradiated MEF feeder layer in DMEM supplemented with 15% fetal bovine serum, 2 mM L-glutamine, 0.1 mM 13-mercaptoethanol, 0.1 mM nonessential amino acids and 1000 U/ml leukaemia inhibitory factor (LIF) (Chemicon; Temecula, Calif.) as previously described in Wang C. et al. (2005) Stem Cells 23:1526-1534. Both HEK and mESCs were incubated at 37° C. in a humidified atmosphere of 95% O₂-5% CO₂.

### Ectopic SID-1 Expression

For transgene delivery, as described in a lentiviral vector (see Trono D. (2002) Lentiviral vectors New York: Spring-Verlag Berlin Heidelberg) was used for modifying human ESCs as described in Wang et al. (2005); Moore J. C. et al. (2005) Reprod Toxicol.

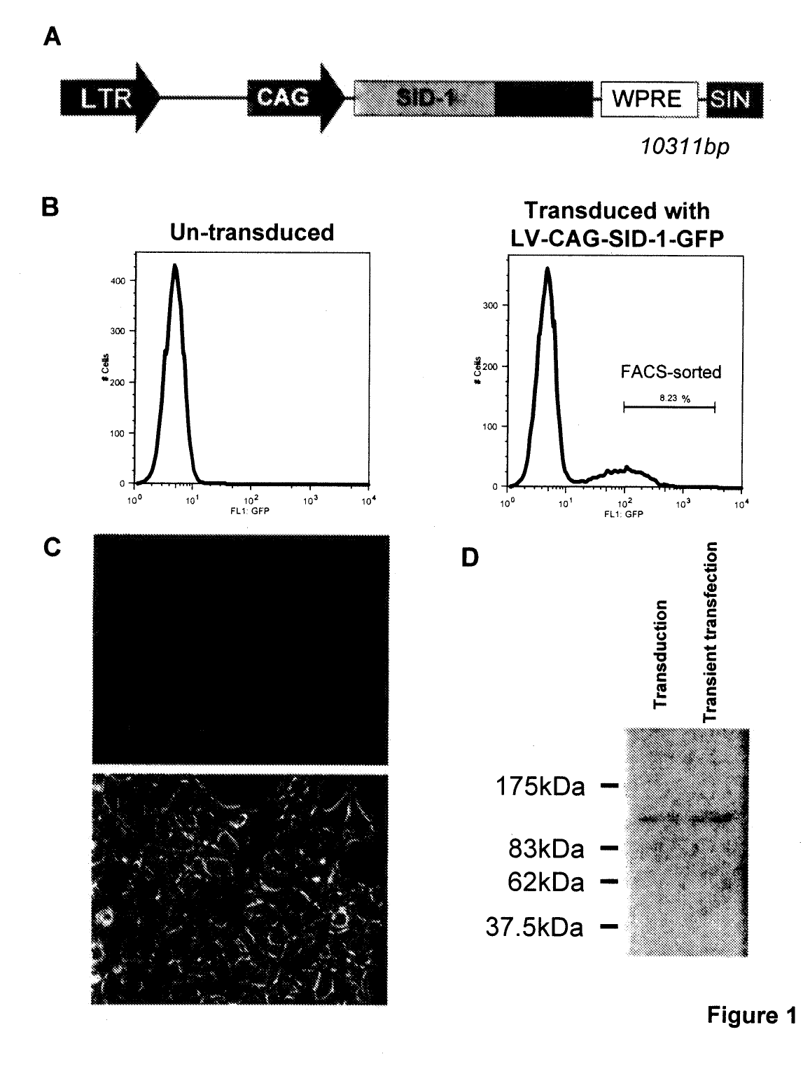

Briefly, the SID-1 transgene from pPACPl-SID-1::FLAG (a kind gift of Dr. Craig P. Hunter, Harvard University) was subcloned into pLV-CAG-GFP to generate pLV-CAG-SID-1-GFP, which directs the expression of the fusion protein SID-1-GFP (with GFP fused to the C-terminus of SID-1) under the control of CAG, an internal composite constitutive promoter containing the CMV enhancer and the β-actin promoter (FIG. 1A). Dr. Hunter has published his characterization of the SID-1 transgene in Hunter et al. (2006) Cold Spring Harbor Symp. Quant. Biol. 71:95-100; Feinberg and Hunter (2003) Science 12:301(5639):1545-1547; and Winston et al. (2002) Science 29:295(5564):2459-2459. CAG was chosen to avoid transgene silencing that is commonly observed in ESCs. Wang et al. (2005); Moore J. C. et al. (2005) Reprod Toxicol, Xue, et al (2005) Circulation 111:11-20. Recombinant lentiviruses (LV) were generated using the 3-plasmid system (see Zufferey R. (1998) J Virol 72:9873-9880) by co-transfecting HEK cells (see below for transfection) with pLV-CAG-GFP or pLV-CAG-SID-1-GFP, pMD.G and pCMVΔR8.91. For each 100-mm dish of HEK cells plated at 80-85% confluency, 5, 2.5, and 10 μg of pLV, pMD.G and pCMVΔR8.91 DNA were used for transfection, respectively. LVs were harvested by collecting the culture medium at 24 and 48 hours post-transfection. LVs generated using this protocol typically had titres in the range of 1×10⁶ to 6×10⁶ TU/mL, and were stored at −80° C. before use. For transduction, purified LVs were added to cells at a final concentration of 10,000 TU ml⁻¹ with 6 μg/mL polybrene. The multiplicity of infection (MOI) was 5. Transduction was allowed to proceed for at least 12-16 hours.

dsRNA and siRNA

dsRNAs (100 bp and 500 bp) that target against firefly luciferase (luc^{Fir}) and Renilla luciferase (luc^{Ren}) were made by in vitro transcription using the Megascript RNAi kit (Ambion; Austin, Tex.). The templates for dsRNA synthesis were gel-purified PCR products from pGL3-luc^{Fir} or pRL-SV40-luc^{Ren} (Promega; Madison, Wis.), respectively. Primers for luc^{Fir} have been described (see Feinberg E. H. et al. (2003) Science 301:1545-1547), and those for luc^{Ren} are provided in Table 1. All primers were purchased from Invitrogen.

TABLE 1
Renilla luciferase (luc^{Ren}) templates:

100 mer

Forward:
luc^{Ren} 100 FORWARD T7:

(SEQ ID NO:_)

TAATACGACTCACTATAGGGCGGCCTCTTCTTATTTATGGC
luc^{Ren} 100 REVERSE:

(SEQ ID NO:_)

GGGCTTGCCTGATTTGCCCATAC
Reverse:
luc^{Ren} 100 REVERSE T7:

(SEQ ID NO:_)

TAATACGACTCACTATAGGGCTTGCCTGATTTGCCCATAC
luc^{Ren} 100 FORWARD:

(SEQ ID NO:_)

GGGCGGCCTCTTCTTATTTATGGC
500 mer
Forward:
luc^{Ren} 500 FORWARD T7

(SEQ ID NO:_)

TAATACGACTCACTATAGGGCGGCCTCTTCTTATTTATGGC
luc^{Ren} 500 REVERSE:

(SEQ ID NO:_)

GGGCGCCATGATAATGTTGGAC
Reverse:
luc^{Ren} 500 FORWARD:

(SEQ ID NO:_)

TAATACGACTCACTATAGGGCGCCATGATAATGTTGGAC
luc^{Ren} 500 REVERSE T7:

(SEQ ID NO:_)

GGGCGGCCTCTTCTTATTTATGGC

For siRNA (Qiagen Inc.; Valencia, Calif.), the sense and antisense sequences were CUUACGCUGAGUACUUCGATT (SEQ ID NO:_) and UCGAAGUACUCAGCGUAAGTT (SEQ ID NO:_) for luc^{Fir}-siRNA, and UUCUCCGAACGUGUCACGUTT (SEQ ID NO:_) and ACGUGACACGUUCGGAGAATT (SEQ ID NO:_) for control (non-silencing) siRNA. All dsRNAs and siRNAs sequences were BLAST searched to ensure no sequence homology to any known mammalian genes.

### Transfection

Transfection was performed using Lipofectamine Plus 2000 (Gibco BRL) according to the manufacturer's protocol. DNA (4 μg/well of 6-well dish) and/or a specified concentration of siRNA or dsRNA were added to cells with lipofectamine. Transfected cells were incubated at 37° C. in a humidified atmosphere of 95% O₂-5% CO₂ for 24 hours for protein expression before luc^{Fir} activities were assessed.

### Flow Cytometry

Flow cytometry analysis was performed on a FACSCalibur instrument (BD Pharmingen; San Diego, Calif.) and analyzed using FlowJo software (Tree Star, San Carlos, Calif.). For assessing apoptosis, Annexin V-Phycoerythrin (Annexin V-PE) and 7-Amino-actinomycin (7-AAD) were employed (BD Pharmingen). Annexin V is a Ca²⁺ dependent phospholipid-binding protein that has a high affinity for phosphatidylserine (PS) which is translocated from the inner to the outer leaflet of plasma membrane during apoptosis. Annexin V conjugated to fluorochromes Phycoerythrin (Annexin V-PE) therefore allows the detection of apoptotic cells by FACS. 7-AAD is a nucleic acid dye which identifies non-viable cells in FACS analysis. Briefly, cells were washed with cold PBS, resuspended in Annexin V-binding buffer (BD Pharmingen) at a concentration of 1×10⁶ cells/mL, followed by incubating with 5 μL of Anexin V-PE and 7-AAD for 15 minutes at room temperature in the dark prior to flow cytometry analysis. For fluorescence-activated cell sorting (FACS), FACS Vantage instrument (BD Pharmingen) was used.

### Western Blot

Cells were harvested in a lysis buffer containing 20 mM HEPES (pH 7.4), 2 mM EGTA, 50 mM β-glycerophosphate, 1 mM Na₃VO₄, 5 mM NaF, 1% Triton X-100, 10% glycerol, 2 mM dithiothreitol, 2 mM phenylmethylsulfonyl fluoride, 2 μg of leupeptin/ml, and 2 μg of aprotinin/ml, vortexed, and centrifuged at 25,000×g for 15 min. Whole-cell protein aliquots (30 μg) were size-fractionated by SDS-PAGE and transferred to nitrocellulose membranes (Schleicher & Schuell; Keene, N. H.). The anti-GFP antibody was purchased from Cell Signaling Technology (Beverly, Mass.). Proteins signals were detected using Western Lightning Chemiluminescence Reagent Plus (Perkin Elmer Life Sciences; Boston, Mass.).

### Luciferase Assay

4×10⁴ cells per well were plated on a solid black 96-well plate (Corning). Luciferase activity was measured using the Steady-Glo luciferase assay system (Promega; Madison, Wis.) on a microplate reader.

### Statistical Analysis

All data reported are means ±S.E.M. Statistical analysis was performed using one-way ANOVA and Tukey's HSD post hoc test with p<0.05 being considered as statistically significant.

### Results

### Generation of a Human Cell Line that Stably Expresses SID-1-GFP

A single round of transduction of HEK cells with LV-CAG-SID-1-GFP yielded 30% GFP-positive cells (vs. 0% for un-transduced control cells; FIG. 1B). Cells that expressed the highest GFP signal, which accounts for 8.23% of the original population, were FACS-sorted, followed by culturing for recovery. Recovered, stably LV-CAG-SID-1-GFP-transduced HEK cells displayed normal morphology (FIG. 1C), remained green for >4 months (longer periods were not monitored) while proliferating not differently from un-transduced control cells (data not shown). Furthermore, GFP epifluorescence was limited to the membrane surface, consistent with the notion that SID-1 is a transmembrane protein. Feinberg E. H. et al. (2003) Science 301:1545-1547. Western blot analysis of stably LV-CAG-SID-1-GFP-transduced HEK cells with a monoclonal antibody against GFP generated a detectable band at 116 kDa consistent with the expected size of SID-1-GFP. Transient pLV-CAG-SID-1-GFP transfection of HEK cells yielded a similar result (FIG. 1D).

dsRNA- and siRNA-Induced Luciferase Suppression was Sequence-Specific

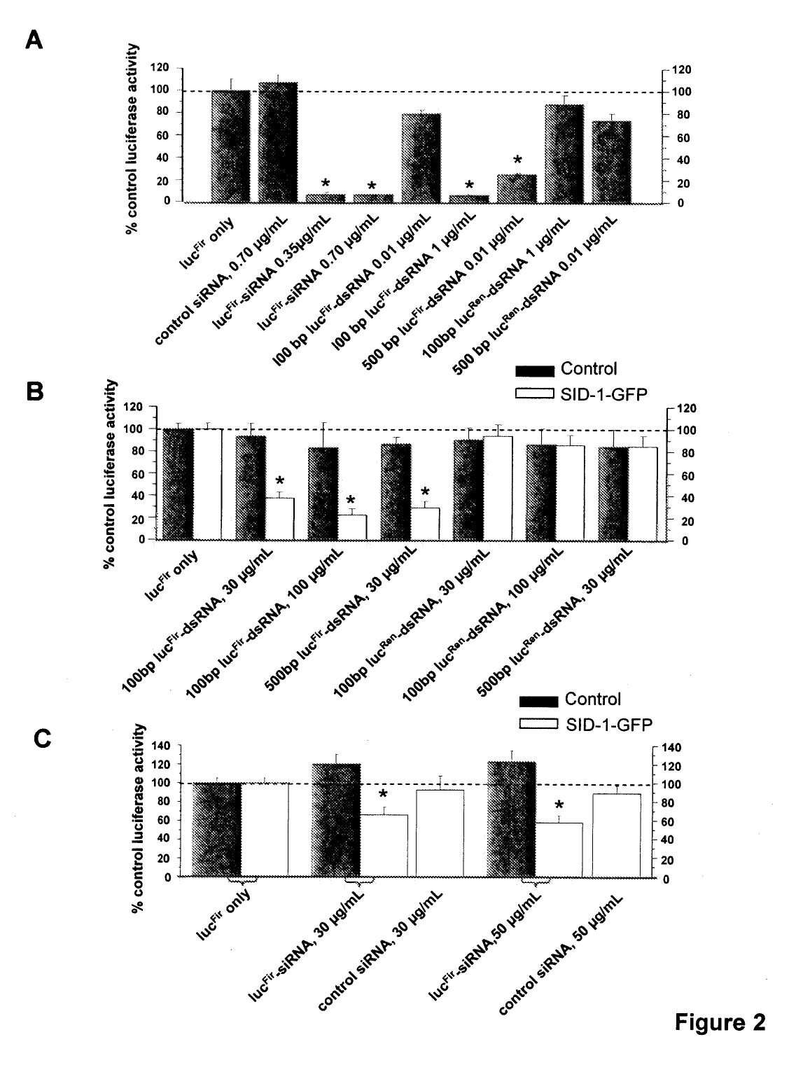

Efficacy of the various siRNA and dsRNA constructs (see Materials and Methods) to induce gene-specific RNAi was verified by assessing their ability to suppress the transiently expressed reporter luc^{Fir} activity. FIG. 2A shows that co-transfection of control HEK cells with pGL3-luc^{Fir}, which encodes for the firefly luciferase (luc^{Fir}), and luc^{Fir}-siRNA, 100 bp or 500 bp luc^{Fir}-dsRNA designed to target luc^{Fir}, significantly suppressed luc^{Fir} activity 24 hrs after transfection (relative to control experiments performed without luc^{Fir}-siRNA or luc^{Fir}-dsRNA; p<0.05). By contrast, control non-silencing siRNA, 100 bp or 500 bp luc^{Ren}-dsRNA that targets Renilla luciferase (luc^{Ren}) had no effect on luc^{Fir} activity (p>0.05), suggesting that the suppressive effects observed with luc^{Fir}-siRNA, 100 bp and 500 bp luc^{Fir}-dsRNA were sequence- and gene-specific.

### LV-CAG-SID-1-GFP-Transduced Cells Uniquely Enabled Passive Entry of Soaking dsRNAs/siRNAs and Subsequent Gene-Specific RNAi

To investigate whether SID-1 overexpression enables passive dsRNA entry into mammalian cells, control (un-transduced) and LV-SID-1-GFP-transduced HEK cells were transfected with pGL3-luc^{Fir}, followed by simple soaking (vs. transfection of FIG. 2A) with dsRNAs (FIG. 2B) or siRNAs (FIG. 2C) for 48 hrs before measuring luc^{Fir} activity. Whereas control cells were insensitive to either luc^{Fir}-dsRNA or luc^{Fir}-siRNA soaking (solid bars) at all the concentrations tested, LV-SID-1-GFP-transduced HEK cells exhibited significantly suppressed luc^{Fir} activity (open bars) under the same conditions: 30 and 100 μg/mL 100 bp luc^{Fir}-dsRNA dose-dependently decreased luc^{Fir} activities to 37.4±5.2% and 22.2±5.9%, respectively (p<0.05; FIG. 2B). When compared with 100 bp luc^{Fir}-dsRNA at the same concentration, 30 μg/mL 500 bp luc^{Fir}-dsRNA induced a modestly stronger (albeit statistically insignificant; FIG. 2B) decrease in luc^{Fir} activity to 28.9±5.9%. Similarly, soaking of SID-1-GFP-expressing cells in luc^{Fir}-siRNAs also led to reduced luc^{Fir} activity (66.3±8.0% and 58.4±7.3% for 30 μg/mL and 50 μg/mL, respectively; p<0.05; FIG. 2C) although the extents of suppression were smaller than those induced by longer luc^{Fir}-dsRNAs at the same concentrations. Collectively, the above observations were in agreement with the notion that SID-1-mediated uptake of dsRNAs or siRNAs (thus, the potency of the resultant RNAi) improves with longer dsRNAs. Feinberg E. H. et al. (2003) Science 301:1545-1547. Of note, luc^{Fir} activity suppression was not observed in the absence of luc^{Fir}-dsRNAs or -siRNAs, or when either control or LV-SID-1-GFP-transduced cells were soaked in control, luc^{Ren}-dsRNA or non-silencing siRNA, suggesting that the SID-1-mediated RNAi effects observed were sequence- and gene-specific.

### Functional Consequences of SID-1 Overexpression in Embryonic Stem Cells

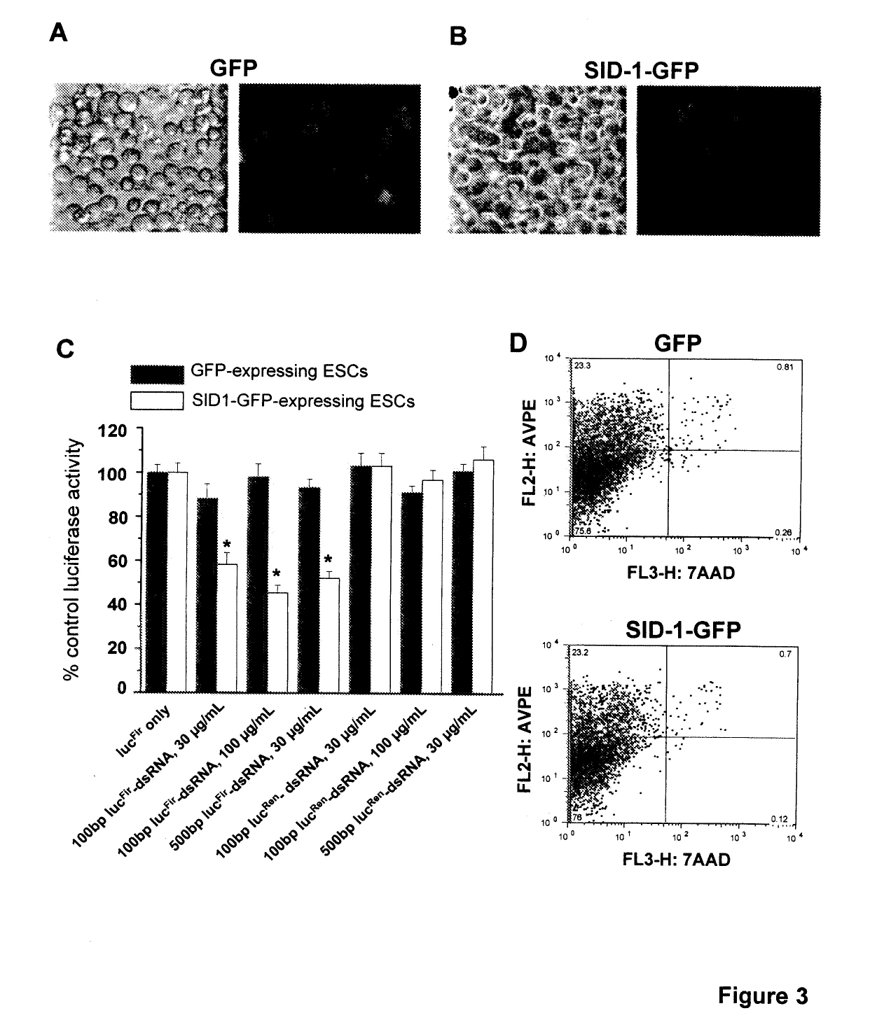

Pluripotent ESCs have the ability to differentiate into all cell types; the ability to conveniently silence multiple genes in undifferentiated ESCs or their tissue-specific derivatives (or progenitors) can facilitate scientific progresses. To explore the use of SID-1 to confer ESCs the ability to passively uptake dsRNA/siRNA so that gene-specific RNAi can be induced by simple soaking, we co-expressed in ESCs luc^{Fir} and either GFP or SID-1-GFP (FIGS. 3A & B). Similar to our HEK cell experiments, only green ESCs were FACS-sorted for subsequent experiments (FIGS. 3C and D). As anticipated, GFP signals were found throughout the entire cytoplasm and limited to the membrane surface for GFP- and SID-1-GFP-expressing ESCs, respectively.

FIG. 3C shows that soaking of SID-1-GFP- but not GFP-expressing ESCs in luc^{Fir}-dsRNA significantly suppressed luc^{Fir} activity. 30 μg/mL and 100 μg/mL 100 bp, and 30 μg/mL 500 bp luc^{Fir}-dsRNAs suppressed the luc^{Fir} activity of SID-1-GFP-expressing cells to 58.3±5.4%, 45.4±3.5% and 52.2±3.1%, respectively (p<0.05). By contrast, no suppression was observed for soaking SID-1-GFP-expressing ESCs 1) without any luc^{Fir}-dsRNA or 2) with 100 bp or 500 bp luc^{Ren}-dsRNA, and 3) control GFP-expressing cells in luc^{Fir}-dsRNA, suggesting that the RNAi-suppressed luc^{Fir} activity observed was SID-1-mediated and sequence-specific. Examination for apoptosis and cell viability by Annexin V-PE and 7-AAD staining revealed no significant differences between GFP- and SID-1-GFP-expressing ESCs (FIG. 3D), suggesting ectopic expression of SID-1 in ESCs led to neither apoptotic nor cytotoxic effects.

### Discussion

SID-1, originally identified in C. elegans, belongs to a novel, uncharacterized gene family that consists of also mammalian homologs. Feinberg E. H. et al. (2003) Science 301:1545-1547, Winston W. M. et al. (2002) Science 295:2456-2459. Although the structure-function properties remain to be better defined, a recent report demonstrates that overexpression of a human SID-1 homolog in pancreatic ductal adenocarcinoma cells enhances the passive uptake of siRNAs. Duxbury M. S. et al. (2005) Biochem Biophys Res Commun 331:459-463. The reported results indicate that the endogenous activity of this human SID-1 homolog, even if expressed, is insignificant in HEK and ES cells because simple soaking with dsRNAs or siRNAs did not lead to RNAi unless when SID-1 is expressed. The lack of detectable endogeneous activity can be attributed to the presence of non-functional SID-1 homologs, functional channel proteins whose activity is suppressed, or they are expressed in a cell type-specific manner. These experiments do not allow us to distinguish between these possibilities. Nonetheless, it has been demonstrated that ectopic SID-1 expression in HEK and ES cells enables passive uptake of dsRNAs and siRNAs, as well as resultant sequence-specific without detrimental effects, at least over the time course of our experiments when parameters such as proliferation rate, morphology and apoptosis, were assessed.

As a form of immune response, accumulation of long dsRNAs (>30 bp), commonly present in numerous viral life cycles, in certain mammalian cells can lead to interferon responses which subsequently block protein translation in a non-specific manner via protein kinase-dependent inactivation of elongation factor, and ultimately, apotosis. Svoboda P. (2004) Cytogenet Genome Res 105:422-434; Kaufman R. J. (1999) Proc Natl Acad Sci USA 96:11693-11695; Der S. D. et al. (1997) Proc Natl Acad Sci USA 94:3279-3283; Sledz C. A. et al. (2004) Biochem Soc Trans 32:952-956. Indeed, the non-specificity of such interferon-meditated effects, which can mask the specific RNAi of interest, have been a major limitation for many RNAi experiments in mammalian cells. Persengiev S. P. (2004) RNA 10:12-18; Sledz C. A. (2003) Nat Cell Biol 5:834-839; Bridge A. J. (2003) Nat Genet. 34:263-264; Moss E. G. (2003) Nat Cell Biol 5:771-772; Kim D. H. (2004) Nat Biotechnol 22:321-325. By contrast, long dsRNA duplexes can efficiently induce specific RNAi in organisms or systems that lack interferon responses. Fire A. et al. (1998) Nature 391:806-811; Svoboda P. (2004) Cytogenet Genome Res 105:422-434; Kennerdell J. R. (1998) Cell 95:1017-1026; Misquitta L. et al. (1999) Proc Natl Acad Sci USA 96:1451-1456; Caplen N. J. (2000) Gene 252:95-105; Ngo H et al. (1998) Proc Natl Acad Sci U S A 95:14687-14692; Billy E. et al. (2001) Proc Natl Acad Sci USA 98:14428-14433; Yang S. et al. (2001) Mol Cell Biol 21:7807-7816; Wianny F. et al. (2000) Nat Cell Biol 2:70-75; Svoboda P. (2000) Development 127:4147-4156; Gan L. (2002) J Neurosci Methods 121:151-157; Yi C. E. (2003) J Biol Chem 278:934-939; Wang J. et al. (2003) Proc Natl Acad Sci USA 100:5103-5106; Svoboda P. et al. (2004) Dev Biol 269:276-285; Paddison P. J. et al. (2002) Proc Natl Acad Sci USA 99:1443-1448. For instance, long dsRNA (typically 500-1500 bp) can readily induce gene-specific silencing in oocytes (see Svoboda P. (2000) Development 127:4147-4156), preimplantation embryos (see Wianny F. et al. (2000) Nat Cell Biol 2:70-75; Svoboda P. et al. (2004) Dev Biol 269:276-285), embryonal teratocarcinoma cells (see Billy E. et al. (2001) Proc Natl Acad Sci USA 98:14428-14433), and ESCs. Yang S. et al. (2001) Mol Cell Biol 21:7807-7816; Paddison P. J. et al. (2002) Proc Natl Acad Sci USA 99:1443-1448. Taken collectively, observations presented herein of gene-specific RNAi with SID-1-expressing ESCs are consistent with the refractoriness of interferons in undifferentiated ESCs to induction. Yang S. et al. (2001) Mol Cell Biol 21:7807-7816; Paddison P. J. et al. (2002) Proc Natl Acad Sci USA 99:1443-1448.

In addition to their promising therapeutic potentials, stem cells and ESCs are also useful models for studying pluripotency, self-renewal and differentiation, etc. Indeed, a better understanding of these important properties and processes of stem cells and ESCs is critical for the development of stem cell-based therapies. Loss-of-function studies have been proven to shed functional and mechanistic insights into various important developmental and differentiation processes. However, high-throughput genomic screening is largely limited by tedious experimental procedures (e.g. homologous recombination to knock-out a specific gene). Gene-specific RNAi has been sought as alternatives for applicable cell types. Transfection or transduction (for transfection-resistant cells such as human ESCs (see Xue, et al (2005) Circulation 111:11-20, Moore J. C. et al. (2005) Reprod Toxicol) and other terminally-differentiated cells such as neurons and cardiomyocytes) is commonly exploited to introduce dsRNAs/siRNAs/shRNAs into cells. Unless delivery vehicles such as those based on lentiviruses and adeno-associated viruses, which require weeks to months to prepare, are employed, however, the resultant RNAi activity is often transient and short-lived. The present results implicate that SID-1-expressing stem cell and ESCs may conveniently enable high-throughput silencing of multiple genes for studying stem cell and ESC differentiation. Such an ability to suppress genes specifically may also lead to the development of novel strategies to inhibit or eliminate the oncogenic potential as well as other potentially adverse effects of stem cell and ESC for therapeutic applications (e.g. suppression of such gene products as ion channels that modulate cell proliferation and/or cause apoptosis). Wang C. et al. (2005) Stem Cells 23:1526-1534, Xue, et al (2005) Circulation 111:11-20, Moore J. C. et al. (2005) Reprod Toxicol.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

What is claimed is:

1. A mammalian stem cell comprising exogenous siRNA or dsRNA polynucleotide and exogenous SID-1 polynucleotide, wherein the cell stably expresses the exogenous SID-1 polypeptide.

2. The mammalian stem cell of claim 1, wherein the cell is a human stem cell.

3. The mammalian stem cell of claim 2, wherein the human stem cell is a somatic stem cell or an embryonic stem cell.

4. The mammalian stem cell of claim 1, wherein the RNA polynucleotide is transcribed from exogenous DNA polynucleotide.

5. The mammalian stem cell of claim 1, wherein the SID-1 polynucleotide is DNA polynucleotide or RNA polynucleotide.

6. The mammalian stem cell of claim 1, wherein the RNA polynucleotide is siRNA.

7. The mammalian stem cell of claim 6, wherein the RNA further comprises a detectable label.

8. The mammalian stem cell of claim 1, wherein the RNA polynucleotide is complementary to a gene having biological activity selected from the group consisting of apoptosis, cell adhesion, cell cycle, immunotherapy, cell signaling, DNA repair and DNA synthesis.

9. The mammalian stem cell of claim 1, wherein the RNA polynucleotide is complementary to a gene having biological activity in immunotherapy.

10. The mammalian stem cell of claim 9, wherein the gene encodes a cytokine, chemokine, transcriptional regulation factor or a translational regulation factor.

11. A method for inducing gene-specific RNA interference in a mammalian stem cell, comprising
introducing an exogenous SID-1 polynucleotide into a mammalian stem cell; and
passively contacting the mammalian stem cell with the gene-specific siRNA or dsRNA in the absence of an insertion vector or carrier such that gene expression for the targeted gene is reduced.

12. The method of claim 11, wherein the mammalian stem cell is a human stem cell.

13. The method of claim 12, wherein the human stem cell is a somatic stem cell or an embryonic stem cell.

14. The method of claim 11, wherein the RNA polynucleotide is siRNA.

15. The method of claim 11, wherein the RNA polynucleotide is complementary to a gene having biological activity selected from the group consisting of apoptosis, cell adhesion, cell cycle, immunotherapy, cell signaling, DNA repair, DNA synthesis.

16. The method of claim 11, wherein the RNA polynucleotide is complementary to a gene having biological activity in immunotherapy.

17. The method of claim 11, wherein the gene encodes a cytokine, chemokine, transcriptional regulation factor or a translational regulation factor.

18. A method of studying stem cell development using RNA interference, comprising introducing an exogenous SID-1 polynucleotide into a mammalian stem cell;
passively contacting the mammalian stem cell with the gene-specific siRNA or dsRNA in the absence of an insertion vector or carrier such that gene expression for the targeted gene is reduced; and comparing the development of the mammalian stem cell to a mammalian stem cell not subjected to RNA interference.

19. The method of claim 18, wherein more than one gene is silenced by siRNA.

20. The method of claim 18, wherein said mammalian stem cell is a human stem cell.

21. The method of claim 19, wherein said human stem cell is a somatic stem cell or an embryonic stem cell.

22. The method of claim 19, wherein the method is performed as a high throughput screen.
